(19) United States
(12) Patent Application Publication
(10) Pub. No.: US 2022/0257612 A1
(43) Pub. Date: Aug. 18, 2022

(54) COMPOSITIONS AND METHODS FOR THE DIAGNOSIS AND TREATMENT OF ALZHEIMER'S DISEASE

(71) Applicants: Rima F. KADDURAH-DAOUK, Durham, NC (US); Siamak MAHMOUDIANDEHKORDI, Durham, NC (US); Cornelia VAN DUIJIN, Oxford (GB); Shahzad AHMAD, Rotterdam (NL); Andrew J. SAYKIN, Indianapolis, IN (US); Kwangsik NHO, Indianapolis, IN (US); Rebecca BAILLIE, San Carlos, CA (US); Xianlin HAN, San Antonio, TX (US); DUKE UNIVERSITY, Durham, NC (US); OXFORD UNIVERSITY, Oxford, (GB); ERASMUS MEDICAL CENTER, Rotterdam (NL); INDIANA UNIVERSITY SCHOOL OF MEDICINE, Indianapolis, IN (US); UNIVERSITY OF TEXAS, San Antonio, TX (US)

(72) Inventors: Rima F. Kaddurah-Daouk, Belmont, MA (US); Siamak Mahmoudiandehkordi, Durham, NC (US); Cornelia van Duijin, Oxford (GB); Shahzad Ahmad, Rotterdam (NL); Andrew J. Saykin, Indianapolis, IN (US); Kwangsik Nho, Indianapolis, IN (US); Rebecca Baillie, San Carlos, CA (US); Xianlin Han, San Antonio, TX (US)

(21) Appl. No.: 17/625,046

(22) PCT Filed: Jul. 24, 2020
(86) PCT No.: PCT/US20/43473

§ 371 Date: Jan. 5, 2022

Related U.S. Application Data
(60) Provisional application No. 62/878,230, filed on Jul. 24, 2019.

(51) Int. Cl. A61K 31/575; A61K 31/40; A61K 31/366; A61K 31/505; A61K 31/216; A61K 31/192; A61K 31/475; A61K 31/427; A61K 31/635; A61K 31/506; A61K 31/27; A61K 31/55; A61K 31/13; A61K 31/445; A61K 31/355; A61K 31/167; A61K 31/07; A61K 31/196; A61K 31/496; A61K 31/60; A61P 25/28

(52) U.S. Cl. CPC A61K 31/575; A61P 25/28; A61K 31/366; A61K 31/505; A61K 31/216; A61K 31/192; A61K 31/475; A61K 31/427; A61K 31/635; A61K 31/506; A61K 31/27; A61K 31/55; A61K 31/13; A61K 31/445; A61K 31/355; A61K 31/167; A61K 31/07; A61K 31/196; A61K 31/496; A61K 31/60; A61K 31/40

(57) ABSTRACT

Described herein are methods for identifying or diagnosing Alzheimer's disease or poor cognition in a subject or population of subjects by analyzing biomarkers. In one aspect, the biomarkers comprise liver function enzymes or metabolites including alanine aminotransferase (ALT), aspartate aminotransferase (AST), ratio of ALT to AST, alkaline phosphatase, albumin, bilirubin, cholesterol and cholesterol metabolites, amino acids, phospholipids, bile acids, or other metabolites. In another aspect, the biomarkers comprise alanine aminotransferase (ALT), aspartate aminotransferase (AST), or the ratio of ALT to AST levels. In another aspect, the marker comprises the genotype of the ALT or AST enzymes.

### CROSS-REFERENCE TO RELATED APPLICATIONS

This application claims priority to U.S. Provisional Patent Application No. 62/878,230, filed on Jul. 24, 2019, which is incorporated by reference herein in its entirety.

### FEDERALLY SPONSORED RESEARCH

This invention was made with United States government support under National Institutes of Health grant numbers: R01AG046171, RF1AG0151550, U01AG024904, U01AG024904-09S4, P50N5053488, R01AG19771, P30AG10133, P30AG10124, K01AG049050, R03AG054936, R01LM011360, R01 LM012535, and R01 EB022574; and Department of Defense Award W81XWH-12-2-0012. The United States government has certain rights in the invention.

### REFERENCE TO SEQUENCE LISTING

This application is filed with a Computer Readable Form of a Sequence Listing in accordance with 37 C.F.R. § 1.821(c). The text file submitted by EFS, “028193-9332-WO01_sequence_listing_23-JUL-2020_ST25.txt,” contains 4 sequences, was created on Jul. 23, 2020, has a file size of 18.4 Kbytes, and is hereby incorporated by reference in its entirety.

### TECHNICAL FIELD

Described herein are methods for identifying or diagnosing Alzheimer's disease or poor cognition in a subject or population of subjects by analyzing biomarkers. In one aspect, the biomarkers comprise liver function enzymes or metabolites including alanine aminotransferase (ALT), aspartate aminotransferase (AST), ratio of ALT to AST, alkaline phosphatase, albumin, bilirubin, cholesterol and cholesterol metabolites, amino acids, phospholipids, bile acids, or other metabolites. In another aspect, the biomarkers comprise alanine aminotransferase (ALT), aspartate aminotransferase (AST), or the ratio of ALT to AST levels. In another aspect, the marker comprises the genotype of the ALT or AST enzymes.

### BACKGROUND

Metabolic activities in the liver determine the state of the metabolic readout of peripheral circulation. Mounting evidence suggests that patients with Alzheimer disease (AD) display metabolic dysfunction [1]. Clinical studies suggest that impaired signaling, energy metabolism, inflammation, and insulin resistance play a role in AD [2, 3]. This observation is in line with the observation that many metabolic disorders (e.g., diabetes, hypertension, obesity, and dyslipidemia) are risk factors for AD [4]. This evidence highlights the importance of the liver in the pathophysiological characteristics of AD. Focused investigation to assess the role of liver function in AD and its endophenotypes is required to bridge the gap between these observations.

Peripheral blood levels of biochemical markers including alanine aminotransferase (ALT), aspartate aminotransferase (AST), albumin, alkaline phosphatase, and total bilirubin are used to assess liver function. Alanine aminotransferase and AST are used in general clinical practice to measure liver injury [5, 6] and are factors associated with cardiovascular and metabolic diseases [7, 8], known risk factors of AD and cognitive decline [9, 10]. Given this fact, it is conceivable that aminotransferases are surrogate biomarkers of liver metabolic functioning. A systematic search yielded few reports related to research in humans linking peripheral biomarkers of liver functioning to central biomarkers related to AD including amyloid-β and tau accumulation, brain glucose metabolism, and structural atrophy.

What is needed are methods for identifying, diagnosing, or treating Alzheimer's disease or poor cognition by analyzing biomarker metabolites and liver enzyme genetic testing in comparison to normal or control subjects.

### SUMMARY

One embodiment described herein is a method for identifying or diagnosing Alzheimer's Disease (AD) including all diseases or disorders that affect normal cognition, memory, perception, learning, and problem solving, including but not limited to Alzheimer's disease, dementia, multi-infarct dementia, dementia with Lewy Bodies, frontotemporal lobar dementia, Huntington's disease, Lewy body disease, traumatic brain injury (TBI), Parkinson's disease, prion disease (Kuru, Creutzfeldt-Jakob disease), dementia/neurocognitive issues due to HIV infection, major neurocognitive disorder, mild neurocognitive disorder, corticobasal syndrome, delirium, amnesia, cognitive dysfunction or poor cognitive performance in a subject or population of subjects, the method comprising: (a) detecting a concentration level in one or more samples from one or more subjects or a population of subjects of one or more biomarkers selected from alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, or a combination thereof, wherein the level of ALT is less than the level in a control sample, or wherein the ratio of AST to ALT levels is greater than the ratio in a control sample; (b) performing a genetic analysis on one or more samples from one or more subjects or a population of subjects to identify mutant, variant, or isozyme ALT, AST, or related genes; and (c) diagnosing one or more subjects as having AD or poor cognitive performance based on the ALT level, AST level, or AST:ALT ratio determined in step (a), or the genetic analysis of the mutant, variant, or isozyme ALT, AST, or related genes identified in step (b).

Another embodiment described herein is a method for stratifying and determining the risk of one or more subjects of a population of subjects for developing Alzheimer's Disease (AD) or poor cognitive performance, the method comprising: (a) detecting a concentration level in one or more samples from one or more subjects of a population of subjects of one or more biomarkers selected from alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, or bile acids, wherein the level of ALT is less than the level in a control sample, or wherein the ratio of AST to ALT levels is greater than the ratio in a control sample; and (b) performing a genetic analysis on one or more samples from one or more subjects of a population of subjects to identify mutant, variant, or isozyme ALT, AST, or related genes; (c) stratifying the levels of alanine aminotransferase (ALT), aspartate aminotransferase (AST), ratio of AST to ALT levels, alkaline phosphatase, albumin, bilirubin, cholesterol and cholesterol metabolites, amino acids, phospholipids, bile acids, and genetic analyses of the mutant, variant, or isozyme ALT, AST, or related genes among the population of subjects to determining subjects at risk of developing AD or poor cognitive performance; and (d) diagnosing subjects of the population of subject as at risk of developing or having AD or poor cognitive performance based on the stratification determined in step (c).

Another embodiment described herein is a method of treating Alzheimer's Disease (AD) or poor cognitive performance in a subject, or population of subjects, the method comprising: (a) detecting a concentration level in one or more samples from one or more subjects or a population of subjects of one or more biomarkers selected from aspartate alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, or bile acids, wherein the level of ALT is less than the level in a control sample, or wherein the ratio of AST to ALT levels is greater than the ratio in a control sample; (b) performing a genetic analysis on one or more samples from one or more subjects or a population of subjects to identify mutant, variant, or isozyme ALT, AST, or related genes; (c) diagnosing one or more subjects or population of subjects as having AD or poor cognitive performance based on the ALT level, AST level, or AST:ALT ratio determined in step (a), or the genetic analysis of the mutant, variant, or isozyme ALT, AST, or related genes identified in step (b); and (d) administering a treatment to the subject(s) determined to have AD or poor cognitive performance. In one aspect, when a population of subjects are evaluated, the method further comprises step (b1) of stratifying the levels of alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, and the genetic analysis of the mutant, variant, or isozyme ALT, AST, or related genes among the population to determining subjects at risk of developing AD or poor cognitive performance.

Another embodiment described herein is a method as described herein wherein the subject or population of subjects are further evaluated using clinical assays, magnetic resonance imaging (MRI), or position emission tomography (PET) for one or more of cerebral spinal fluid (CSF) amyloid-β1-42 levels; amyloid-β deposition; CSF phosphorylated tau levels; CSF total tau levels; brain glucose metabolism; brain atrophy, or a combination thereof. In one aspect, the subject or population of subjects diagnosed as having or at risk for AD or poor cognitive performance has one or more of: lower cerebral spinal fluid (CSF) amyloid-β1-42 levels; increased amyloid-β deposition; greater CSF phosphorylated tau levels; greater CSF total tau levels; reduced brain glucose metabolism; greater brain atrophy; or a combination thereof.

Another embodiment described herein is the administration of a treatment to the subject(s) determined to have a risk of developing or having AD or poor cognitive performance. In one aspect, the treatment comprises one or more of: bile acids (chenodeoxycholic acid (CDCA), cholic acid, ursodiol, tauroursodeoxycholic acid, ursodeoxycholic acid, obeticholic acid, glycocholic acid); bile acid sequestrants (Cholestyramine, Colesevelam, Colestilan, Colestipol, Ezetimibe); Statins (Atorvastatin, Lovastatin, Rosuvastatin, Simvastatin); fibrates (Fenofibrate); Heal Bile Acid Transporter (IBAT) inhibitors (Volixibat, Odevixibat, Elobixibat, Maralixibat, Albireo Pharma unnamed compounds); farnesoid X receptor agonists (Tropifexor, Cilofexor, EYP001a, GW4064, cafestol, chenodeoxycholic acid, obeticholic acid (OCA), Fexaramine, INT-767, Px-104, EDP-305, Gilead unnamed compounds; Metacrine unnamed compounds); G-protein-coupled bile acid receptor TRG5 agonists (6-EMCS, INT-777, Ardelyx unnamed compounds, Zydus Research Centre unnamed compounds); peroxisome proliferator-activated receptor (PPAR) agonists (Elafibranor GFT505); or Multidrug Resistance (MDR) Inhibitors (Vinblastine, Ritonavir, Furosemide, Lamivudine). In another aspect, the treatment comprises or further comprises one or more of: rivastigmine (Exelon®), galantamine (Razadyne®), memantine (Namenda®), a combination of memantine and donepezil (Namzaric®); antidepressants comprising citalopram (Celexa®), escitalopram (Lexapro®), fluoxetine (Prozac®), paroxetine (Paxil®), sertraline (Zoloft®), or trazodone (Desyrel®); anxiolytics comprising lorazepam (Ativan®) or oxazepam (Serax®); antipsychotic comprising aripiprazole (Abilify®), clozapine (Clozaril®), haloperidol (Haldol®), olanzapine (Zyprexa®), quetiapine (Seroquel®), risperidone (Risperdal®), or ziprasidone (Geodon®); tricyclic antidepressants comprising amitriptyline, amoxapine, desipramine (Norpramin®), doxepin, imipramine (Tofranil®), nortriptyline (Pamelor®), protriptyline, trimipramine; benzodiazepines comprising lorazepam, oxazepam or temazepam; sleeping treatments comprising zolpidem (Ambien®), zaleplon (Sonata®), eszopiclone (Lunesta®), phenobarbital, or chloral hydrate; atypical antipsychotics comprising risperidone, olanzapine, or quetiapine; classical antipsychotics comprising haloperidol; non-steroidal antiinflammatory drugs (NSAIDs, ibuprofen, naproxen, diclofenac, acetylsalicylic acid), acetaminophen, or alternative treatments or dietary supplements comprising amino acids (alanine, aspartate, glutamate, etc.), α-ketoglutarate, pyridoxal phosphate, vitamins (retinol (A), thiamine (B1), riboflavin (B2), niacinamide (B3), adenine (B4), pantothenic acid (B5), pyridoxine (B6), biotin (B7), adenylate (B8), carnitine (BT), folic acid (B9), cobalamin (B12), ascorbic acid (C), cholecalciferol (D), tocopherol (E), essential fatty acids (F), catechol (J), phylloquinone (K), salicylic acid (S), S-methylmethionine (U), inositol, choline), huperzine A, tramiprosate, caprylic acid, coconut oil, omega-3 fatty acids (fish oil, Lovaza®, Vascepa®, Epanova®, Omtryg®, Vscazen®), coenzyme Q10, phosphatidylserine, coral calcium, or Ginkgo biloba extracts.

In another embodiment described herein, the subject or population of subjects has liver disease. In one aspect, the subject or population of subjects has decreased liver function. In another aspect, the control sample is from a subject or population of subjects with normal cognition. In another aspect, the control sample is from a subject or population of subjects not having AD or poor cognition. In another aspect, the control sample is from a subject or population of subjects not having liver disease. In another aspect, the sample comprises whole blood, serum, plasma, or cerebral spinal fluid (CSF). In another aspect, the sample comprises blood. In another aspect, the sample comprises cerebral spinal fluid (CSF).

Another embodiment described herein is the use of one or more biomarkers selected from alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, or a combination thereof and genetic analysis to identify mutant, variant, or isozyme ALT, AST, or related genes for identifying or diagnosing Alzheimer's Disease (AD) or poor cognitive performance in a subject or population of subjects.

Another embodiment described herein is the use of one or more biomarkers selected from alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, or a combination thereof and genetic analysis to identify mutant, variant, or isozyme ALT, AST, or related genes for stratifying a population of subjects for determining risk of developing or having Alzheimer's Disease (AD) or poor cognitive performance.

Another embodiment described herein is the use of one or more biomarkers selected from alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, or a combination thereof and genetic analysis to identify mutant, variant, or isozyme ALT, AST, or related genes for determining the risk of a subject or population of subjects developing Alzheimer's Disease (AD) or poor cognitive performance.

Another embodiment described herein is the use of one or more biomarkers selected from alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, or a combination thereof and genetic analysis to identify mutant, variant, or isozyme ALT, AST, or related genes for identifying or diagnosing Alzheimer's Disease (AD) or poor cognitive performance in a subject or population of subjects; and administering a treatment to the subject determined to have AD or poor cognitive performance.

Another embodiment described herein is a use as described herein wherein the subject or population of subjects are further evaluated using clinical assays, magnetic resonance imaging (MRI), or position emission tomography (PET) for one or more of cerebral spinal fluid (CSF) amyloid-β1-42 levels; amyloid-β deposition; CSF phosphorylated tau levels; CSF total tau levels; brain glucose metabolism; brain atrophy, or a combination thereof. In one aspect, the subject or population of subjects diagnosed as having or at risk for AD or poor cognitive performance has one or more of: lower cerebral spinal fluid (CSF) amyloid-β1-42 levels; increased amyloid-β deposition; greater CSF phosphorylated tau levels; greater CSF total tau levels; reduced brain glucose metabolism; greater brain atrophy; or a combination thereof.

Another embodiment described herein is the use further comprising the administration of a treatment to the subject(s) determined to have a risk of developing or having AD or poor cognitive performance.

### DESCRIPTION OF THE DRAWINGS

The patent or application contains at least one drawing executed in color. Copies of this patent application publication or patent with color drawing(s) will be provided by the Office upon request and payment of the necessary fee.

FIG. 1 shows a heat map of q-values of the association between liver function markers and the A/T/N biomarkers for Alzheimer disease. P values estimated from linear regression analyses were corrected for multiple testing using false discovery rate (q-value). White indicates q>0.05, red indicates significant positive association, and green indicates significant negative association. AR indicates amyloid-β; ALT, alanine aminotransferase; AST, aspartate aminotransferase; CSF, cerebrospinal fluid; FDG, fludeoxyglucose positron emission tomography; MRI, magnetic resonance imaging; and p-tau, phosphorylated tau.

FIG. 2 shows whole-brain multivariable analysis was performed to visualize the topography of the association of ALT levels and AST to ALT ratio values with amyloid-β load and glucose metabolism on a voxelwise level (false discovery rate−corrected P<0.05). FIG. 2A shows higher ALT levels were significantly associated with reduced amyloid-β deposition in the bilateral parietal lobes. FIG. 2B shows increased ALT levels were significantly associated with increased glucose metabolism in a widespread manner, especially in the bilateral frontal, parietal, and temporal lobes. FIG. 2C shows increased AST to ALT ratio values were significantly associated with increased amyloid-β deposition in the bilateral parietal lobes and the right temporal lobe. FIG. 2D shows increased AST to ALT ratio values were significantly associated with reduced brain glucose metabolism in the bilateral frontal, parietal, and temporal lobes.

FIG. 3 shows a whole-brain multivariable analysis of cortical thickness across the brain surface was performed to visualize the topography of the association of ALT levels with brain structure. Statistical maps were thresholded using a random field theory for a multiple testing adjustment to a corrected significance level of P<0.05. The P value for clusters indicates significant corrected P values with the lightest blue color. Higher ALT levels were significantly associated with greater cortical thickness, especially in bilateral temporal lobes.

FIG. 4 shows liver function biomarkers and their association with ATN biomarkers for Alzheimer's disease adjusted for statin use and gamma-glutamyltransferase. Heat map of q-values of association between liver function biomarkers and the “A/T/N” biomarkers for Alzheimer's disease. P-values estimated from linear regression analyses were corrected for multiple testing using FDR (q-value). Color code: white indicates q-value>0.05, red indicates significant positive associations, and green indicates significant negative associations.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting. Further, unless otherwise required by context, singular terms shall include pluralities and plural terms shall include the singular.

As used herein the terms “comprise(s),” “include(s),” “having,” “has,” “can,” “contain(s),” and variants thereof are intended to be open-ended transitional phrases, terms, or words that do not preclude the possibility of additional acts or structures. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein “Alzheimer's Disease” includes all diseases or disorders that affect normal cognition, memory, perception, learning, and problem solving, including but not limited to Alzheimer's disease, dementia, multi-infarct dementia, dementia with Lewy Bodies, frontotemporal lobar dementia, Huntington's disease, Lewy body disease, traumatic brain injury (TBI), Parkinson's disease, prion disease (Kuru, Creutzfeldt-Jakob disease), dementia/neurocognitive issues due to HIV infection, major neurocognitive disorder, mild neurocognitive disorder, corticobasal syndrome, delirium, amnesia, and cognitive dysfunction.

As used herein the terms “subject” and “patient” interchangeably refer to any vertebrate, including, but not limited to, a mammal and a human. In some embodiments, the subject may be a human or a non-human. The subject or patient may be undergoing forms of treatment. “Mammal” as used herein refers to any member of the class Mammalia, including, without limitation, humans and nonhuman primates such as chimpanzees and other apes and monkey species; farm animals such as cattle, sheep, pigs, goats, llamas, camels, and horses; domestic mammals such as dogs and cats; laboratory animals including rodents such as mice, rats, rabbits, guinea pigs, and the like. The term does not denote a particular age or sex. Fetuses, neonates, juveniles, adolescents, adults, or geriatrics whether male or female, are intended to be included within the scope of this term.

As used herein, “sample,” “test sample,” and “biological sample” refer to fluid sample containing or suspected of containing a biomarker metabolite. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing a biomarker metabolite may be assayed directly. In one embodiment, the source containing a biomarker metabolite is a human bodily substance (e.g., bodily fluid, blood such as whole blood, serum, plasma, urine, saliva, sweat, sputum, semen, mucus, lacrimal fluid, lymph fluid, amniotic fluid, interstitial fluid, lung lavage, cerebrospinal fluid, feces, tissue, organ, or the like). Tissues may include, but are not limited to skeletal muscle tissue, liver tissue, lung tissue, kidney tissue, myocardial tissue, brain tissue, bone marrow, cervix tissue, skin, etc. The sample may be a liquid sample or a liquid extract of a solid sample. In certain cases, the source of the sample may be an organ or tissue, such as a biopsy sample, which may be solubilized by tissue disintegration/cell lysis. In one aspect, the sample is blood, a blood fraction, or cerebrospinal fluid.

As used herein, “genetic testing,” or “genetic analysis” refer to sequencing or other methods for determining the genetic sequence of a particular gene (or genes) to determine the “genotype.” In one embodiment, the genotype of the liver ALT and AST enzymes is determined in conjuction with liver function tests to evaluate whether a genetic mutation, variant, isozyme, or other genetic aberration may be the source of atypical liver function test results.

As used herein, the terms “treat”, “treating,” or “treatment” of any disease or disorder refer In an embodiment, to ameliorating the disease or disorder (i.e., slowing or arresting or reducing the development of the disease or at least one of the clinical symptoms thereof). In an embodiment, “treat,” “treating,” or “treatment” refers to alleviating or ameliorating at least one physical parameter including those which may not be discernible by the patient.

As used herein, the term “preventing” refers to a reduction in the frequency of, or delay in the onset of, symptoms of the condition or disease.

As used herein, a subject is “in need of” a treatment if such subject would benefit biologically, medically, or in quality of life from such treatment.

As used herein, the term “prophylaxis” refers to preventing or reducing the progression of a disorder, either to a statistically significant degree or to a degree detectable to one skilled in the art.

As used herein the term “substantially” means to a great or significant extent, but not completely.

As used herein, all percentages (%) refer to mass (or weight, w/w) percent unless noted otherwise.

As used herein the term “about” refers to any values, including both integers and fractional components that are within a variation of up to ±10% of the value modified by the term “about.”

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified. As used herein the terms “include,” “including,” “contain,” “containing,” “having,” and the like mean “comprising.”

As used herein the term “or” can be conjunctive or disjunctive.

The liver plays many crucial roles in normal brain metabolism and functions. For example, ketone bodies are specifically produced in the liver and the liver plays a key role in maintaining normal blood glucose homeostasis, whereas both glucose and ketone bodies are the major energy substrates for the brain. Moreover, the liver produces many lipids (e.g., essential fatty acids) and/or lipid precursors (e.g., precursors for plasmalogen biosynthesis) for the brain to maintain normal brain integrity and function. Therefore, injuries or diseases of liver can cause brain energy imbalances and deficiencies in some brain lipids, and consequently affect brain metabolism, integrity, and functions. Chronic liver disease and injuries to the liver can lead to the loss of brain function, cognitive decline, and dementia. By better understanding the liver-brain metabolic axis, prevention and treatment regimens can be developed for AD and cognitive decline.

Non-alcoholic fatty liver disease (NAFLD) is a chronic characterized by excessive fat deposition in hepatocytes. NAFLD ranges from simple steatosis, or fatty liver, to nonalcoholic steatohepatitis (NASH) to cirrhosis occurring frequently in conjunction with obesity, dyslipidemia, and insulin resistance. NAFLD is associated with insulin resistance, though whether insulin resistance causes hepatic steatosis or whether hepatic steatosis per se reduces insulin sensitivity is unclear. Development of hepatic steatosis has been linked to consumption of a high fat diet, obesity, alterations in hepatic and whole-body lipid metabolism, and increases in inflammation and reactive oxygen species. NAFLD has been associated with depression, anxiety, cognitive functioning, and Alzheimer's Disease. In particular, patients with NAFLD are more likely to develop depression and anxiety.

Treatment of NAFLD and NASH often involves treatment to reduce insulin resistance, lipid accumulation, and inflammation. These treatments include life-style changes as well as anti-diabetic medications, including, GLP-1 agonists, PPARγ agonists, and metformin. Bile acids and bile acid derivatives have been used to reduce cholesterol and lipids. Other medications have been developed or are in development to reduce lipid accumulation and inflammation. These drugs include PPARα agonists, PPARδ agonists, CCR2 antagonists, FGF21 agonists, phosphodiesterase inhibitors, and AMPK agonists. All of these drugs have the potential to improve cognitive function.

Other liver diseases associated with depression, anxiety, or Alzheimer's Disease include chronic liver disease, alcoholic liver disease, cirrhosis, and autoimmune liver disease. Any chronic liver disease which reduces liver function or destroys liver cells.

Drug categories and drugs that are used to treat liver disease comprise: bile acids (chenodeoxycholic acid (CDCA), cholic acid, ursodiol, tauroursodeoxycholic acid, ursodeoxycholic acid, obeticholic acid, glycocholic acid); bile acid sequestrants (Cholestyramine, Colesevelam, Colestilan, Colestipol, Ezetimibe); Statins (Atorvastatin, Lovastatin, Rosuvastatin, Simvastatin); fibrates (Fenofibrate); Ileal Bile Acid Transporter (IBAT) inhibitors (Volixibat, Odevixibat, Elobixibat, Maralixibat, Albireo Pharma unnamed compounds); farnesoid X receptor agonists (Tropifexor, Cilofexor, EYP001a, GW4064, cafestol, chenodeoxycholic acid, obeticholic acid (OCA), Fexaramine, INT-767, Px-104, EDP-305, Gilead unnamed compounds; Metacrine unnamed compounds); G-protein-coupled bile acid receptor TRG5 agonists (6-EMCS, INT-777, Ardelyx unnamed compounds, Zydus Research Centre unnamed compounds); peroxisome proliferator-activated receptor (PPAR) agonists (Elafibranor GFT505); or Multidrug Resistance (MDR) Inhibitors (Vinblastine, Ritonavir, Furosemide, Lamivudine). There are additional drugs in each category and additional drugs in development.

Liver injury can be determined by measuring AST and ALT levels and their ratios. Therefore, the measurement of these parameters can provide a means to evaluate brain function and cognition to a certain degree and can be used to phenotype causal factors for cognitive problems. These types of measurements permit personalized, specific treatments for subjects with drug(s) targeted to these enzymes or related enzymes in the metabolic pathway and the supplementation of particular metabolites such as glucose, amino acids, medium chain triglycerides, acetyl carnitine, BCAA, ketone bodies, among other metabolites. Further, this phenotype/genotype methodology permits the stratification or sub-stratification of populations of subjects to identify subjects at risk of developing AD or cognitive decline and potentially abrogate this risk by precision-medicine drug administration or supplementation therapy.

Embodiments described herein relate generally to the analysis and identification of global metabolic changes in Alzheimer's disease (AD). More particularly, materials and methods relating to the use of metabolomics as a biochemical approach to identify peripheral metabolic changes in AD patients and correlate them to cerebrospinal fluid pathology markers, imaging features, and cognitive performance are described herein. In addition, genotyping can be used to identify mutations and variants in specific metabolic pathways associated with liver disease.

Described herein are methods for evaluating the correlation between liver enzyme levels and neuroimaging changes of the brain associated with AD. These methods can be used to stratify subjects by the liver enzyme levels, their ratios and define subgroups affected or at risk for AD or cognitive decline. Affected subjects can be administered drug or supplement therapies to prevent the onset, ameliorate the symptoms, or treat both liver disease and/or AD or cognitive decline.

The methods described herein permit the determination of a subject or population of subjects' “metabotype” by measuring the levels of particular enzymes and metabolite biomarkers in the blood or cerebrospinal fluid. In addition, mutants, variants, and isotypes of specific enzymes in metabolic pathways associated with liver disorders can be identified by determining their genotype through sequence analyses. By understanding both the metabotype and genotype of the liver's metabolic processes, the risks associated with AD or cognitive decline or the severity thereof can be determined. In addition, if treatment or preventative measures are indicated, personalized treatment regimens can be developed using drug therapy and metabolite supplements (e.g., amino acids, co-factors, etc.) to facilitate the effected metabolic pathways and supplement their precursors, substrates, co-factors, or products.

The association and correlation of peripheral liver function markers with AD diagnosis, cognition, and biomarkers of AD pathophysiological characteristics including neuroimaging (magnetic resonance imaging (MRI) and position emission tomography (PET) and cerebrospinal fluid (CSF) from older adults in the AD Neuroimaging Initiative (ADNI) cohort was investigated. The AD biomarkers were selected and defined consistent with the National Institute on Aging-Alzheimer Association Research Framework (e.g., amyloid, tau, and neurodegeneration (A/T/N)) for AD biomarkers that defines 3 general groups of biomarkers based on the nature of pathologic process that each measures [11]. See [79]: Nho et al., JAMA Netw Open 2(7):e197978 (2019), which is incorporated by reference herein for such teachings.

In one embodiment, a sample is obtained from a subject or population of subjects. Typically, the sample is whole blood, serum, plasma, and/or cerebral spinal fluid (CSF). The sample can be analyzed using standard clinical laboratory testing for the presence of biomarker metabolites. For example, a complete blood count (CBC), basic metabolic panel (BMP), comprehensive metabolic panel (CMP), or lipid panel (LP) can be performed. In some embodiments, additional specific tests are performed on the sample to assay the levels of particular metabolites that are not part of the typical clinical laboratory panels. In one embodiment, the biomarkers comprise concentration levels of liver function enzymes or metabolites including alanine aminotransferase (ALT), aspartate aminotransferase (AST), ratio of ALT to AST, alkaline phosphatase, albumin, bilirubin, cholesterol and cholesterol metabolites, amino acids, phospholipids, bile acids, or other metabolites. The results of such tests are reviewed and can be used as described herein to stratify, sub-stratify, identify, diagnose, or determine whether a subject or population of subjects has or is at risk of developing Alzheimer's Disease (AD) or poor cognitive performance.

Alanine aminotransferase (ALT) is an enzyme found mostly in the cells of the liver and kidney. The function of ALT is to convert alanine into pyruvate, an important intermediate in glycolysis and the TCA cycle. ALT catalyzes the transfer of an amino group from L-alanine to α-ketoglutarate, the products of this reversible transamination reaction being pyruvate and L-glutamate. ALT (and all aminotransferases) requires the coenzyme pyridoxal phosphate, which is converted into pyridoxamine in the first step of the reaction, when an amino acid is converted into a keto acid. The reaction catalyzed by ALT is shown in Scheme 1:

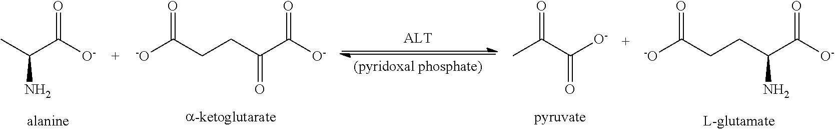

Similarly, aspartate transaminase (AST) catalyzes the interconversion of L-aspartate and α-ketoglutarate to oxaloacetate and L-glutamate. The reaction catalyzed by AST is shown in Scheme 2:

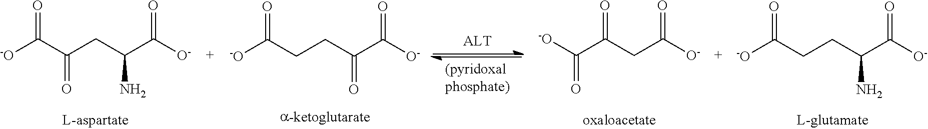

Clinical alanine aminotransferase (ALT) and aspartate aminotransferase (AST) tests measure the concentration levels of ALT and AST in the blood and are useful for the detection of liver disease. In healthy individuals, ALT and AST levels in the blood are low. Typical ALT reference range values for normal subjects are 45 IU/L for males and 34 IU/L for females. Typical AST reference range values for normal subjects are 8-40 IU/L for males and 6-34 IU/L for females.

AST is similar to ALT in that both enzymes are associated with liver parenchymal cells. The difference is that ALT is found predominantly in the liver, with clinically negligible quantities found in the kidneys, heart, and skeletal muscle, while AST is found in the liver, heart (cardiac muscle), skeletal muscle, kidneys, brain, and red blood cells. As a result, ALT is a more specific indicator of liver inflammation than AST, because AST may be elevated also in diseases affecting other organs, such as myocardial infarction, acute pancreatitis, acute hemolytic anemia, severe burns, acute renal disease, musculoskeletal diseases, and trauma.

When the liver is damaged, both ALT and AST are released into the blood, usually before more obvious signs of liver damage occur, such as jaundice. When elevated ALT and AST levels are found in the blood, the possible underlying causes can be further narrowed down by measuring other enzymes. For example, elevated ALT levels due to hepatocyte damage can be distinguished from bile duct problems by measuring alkaline phosphatase. Also, myopathy-related elevations in ALT should be suspected when the AST level is greater than ALT; the possibility of muscle disease causing elevations in liver tests can be further explored by measuring muscle enzymes, including creatine kinase.

A calculated AST:ALT ratio is useful for differentiating between different causes of liver injury and in recognizing when the increased levels may be coming from another source, such as heart or muscle injury. The AST:ALT ratio increases in liver functional impairment. In alcoholic liver disease, the mean ratio is 1.45, and mean ratio is 1.33 in post necrotic liver cirrhosis. The ratio is greater than 1.17 in viral cirrhosis, greater than 2.0 in alcoholic hepatitis, and 0.9 in non-alcoholic hepatitis. The ratio is greater than 4.5 in Wilson disease or hyperthyroidism.

A number of conditions can cause injury to liver cells and may cause increases in ALT or AST. The test is most useful in detecting liver damage due to hepatitis, drugs toxic to the liver, cirrhosis, or alcoholism. Many drugs may also elevate ALT levels, including zileuton, omega-3 fatty acid ethyl esters (Lovaza®), anti-inflammatory drugs (e.g., NASIDs), antibiotics, cholesterol medications, some antipsychotics such as risperidone, and anticonvulsants. Acetaminophen may also elevate ALT levels.

Genetic analyses can also be used in conjunction with liver enzyme tests to determine whether the genes encoding ALT and AST enzymes encode normal wild type enzymes, isozymes, variants, or mutants. Also enzymes involved in their level regulation might be impacted. Typical genetic sequencing can be used for such analyses. The locus for the ALT1 gene is 8q24.3 (see NCBI Gene 2875). The wild type human ALT enzyme has the nucleotide and polypeptide sequence provided in NCBI Accession No. NP_001369594 (i.e., SEQ ID NOs: 1-2, for the CDS nucleotide and polypeptide sequences, respectively). The locus for AST gene (glutamic-oxaloacetic transaminase 1, GOT1 is 10q24.2 (see NCBI Gene 2805). The wilde type human AST nucleotide and polypeptide sequence provided in GenBank Accession No. EAW49868.1 (i.e., SEQ ID NOs: 3-4, for the CDS nucleotide and polypeptide sequences, respectively).

One embodiment described herein is a method for identifying or diagnosing Alzheimer's Disease (AD) or poor cognitive performance in a subject or population of subjects, the method comprising: (a) detecting a concentration level in one or more samples from one or more subjects or a population of subjects of one or more biomarkers selected from alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, or a combination thereof, wherein the level of ALT is less than the level in a control sample, or wherein the ratio of AST to ALT levels is greater than the ratio in a control sample; (b) performing a genetic analysis on one or more samples from one or more subjects or a population of subjects to identify mutant, variant, or isozyme ALT, AST, or related genes; and (c) diagnosing one or more subjects as having AD or poor cognitive performance based on the ALT level, AST level, or AST:ALT ratio determined in step (a), or the genetic analysis of the mutant, variant, or isozyme ALT, AST, or related genes identified in step (b).

Another embodiment described herein is a method for stratifying and determining the risk of one or more subjects of a population of subjects for developing Alzheimer's Disease (AD) or poor cognitive performance, the method comprising: (a) detecting a concentration level in one or more samples from one or more subjects of a population of subjects of one or more biomarkers selected from alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, or bile acids, wherein the level of ALT is less than the level in a control sample, or wherein the ratio of AST to ALT levels is greater than the ratio in a control sample; and (b) performing a genetic analysis on one or more samples from one or more subjects of a population of subjects to identify mutant, variant, or isozyme ALT, AST, or related genes; (c) stratifying the levels of alanine aminotransferase (ALT), aspartate aminotransferase (AST), ratio of AST to ALT levels, alkaline phosphatase, albumin, bilirubin, cholesterol and cholesterol metabolites, amino acids, phospholipids, bile acids, and genetic analyses of the mutant, variant, or isozyme ALT, AST, or related genes among the population of subjects to determining subjects at risk of developing AD or poor cognitive performance; and (d) diagnosing subjects of the population of subject as at risk of developing or having AD or poor cognitive performance based on the stratification determined in step (c).

Another embodiment described herein is a method of treating Alzheimer's Disease (AD) or poor cognitive performance in a subject, or population of subjects, the method comprising: (a) detecting a concentration level in one or more samples from one or more subjects or a population of subjects of one or more biomarkers selected from aspartate alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, or bile acids, wherein the level of ALT is less than the level in a control sample, or wherein the ratio of AST to ALT levels is greater than the ratio in a control sample; (b) performing a genetic analysis on one or more samples from one or more subjects or a population of subjects to identify mutant, variant, or isozyme ALT, AST, or related genes; (c) diagnosing one or more subjects or population of subjects as having AD or poor cognitive performance based on the ALT level, AST level, or AST:ALT ratio determined in step (a), or the genetic analysis of the mutant, variant, or isozyme ALT, AST, or related genes identified in step (b); and (d) administering a treatment to the subject(s) determined to have AD or poor cognitive performance. In one aspect, when a population of subjects are evaluated, the method further comprises step (b1) of stratifying the levels of alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, and the genetic analysis of the mutant, variant, or isozyme ALT, AST, or related genes among the population to determining subjects at risk of developing AD or poor cognitive performance.

Another embodiment described herein is a method as described herein wherein the subject or population of subjects are further evaluated using clinical assays, magnetic resonance imaging (MRI), or position emission tomography (PET) for one or more of cerebral spinal fluid (CSF) amyloid-β1-42 levels; amyloid-β deposition; CSF phosphorylated tau levels; CSF total tau levels; brain glucose metabolism; brain atrophy, or a combination thereof. In one aspect, the subject or population of subjects diagnosed as having or at risk for AD or poor cognitive performance has one or more of: lower cerebral spinal fluid (CSF) amyloid-β1-42 levels; increased amyloid-β deposition; greater CSF phosphorylated tau levels; greater CSF total tau levels; reduced brain glucose metabolism; greater brain atrophy; or a combination thereof.

Another embodiment described herein is the administration of a treatment to the subject(s) determined to have a risk of developing or having AD or poor cognitive performance. In one aspect, the treatment comprises one or more of: bile acids (chenodeoxycholic acid (CDCA), cholic acid, ursodiol, tau roursodeoxycholic acid, ursodeoxycholic acid, obeticholic acid, glycocholic acid); bile acid sequestrants (Cholestyramine, Colesevelam, Colestilan, Colestipol, Ezetimibe); Statins (Atorvastatin, Lovastatin, Rosuvastatin, Simvastatin); fibrates (Fenofibrate); Ileal Bile Acid Transporter (IBAT) inhibitors (Volixibat, Odevixibat, Elobixibat, Maralixibat, Albireo Pharma unnamed compounds); farnesoid X receptor agonists (Tropifexor, Cilofexor, EYP001a, GW4064, cafestol, chenodeoxycholic acid, obeticholic acid (OCA), Fexaramine, INT-767, Px-104, EDP-305, Gilead unnamed compounds; Metacrine unnamed compounds); G-protein-coupled bile acid receptor TRG5 agonists (6-EMCS, INT-777, Ardelyx unnamed compounds, Zydus Research Centre unnamed compounds); peroxisome proliferator-activated receptor (PPAR) agonists (Elafibranor GFT505); or Multidrug Resistance (MDR) Inhibitors (Vinblastine, Ritonavir, Furosemide, Lamivudine). In another aspect, the treatment comprises or further comprises one or more of: rivastigmine (Exelon®), galantamine (Razadyne®), memantine (Namenda®), a combination of memantine and donepezil (Namzaric®); antidepressants comprising citalopram (Celexa®), escitalopram (Lexapro®), fluoxetine (Prozac®), paroxetine (Paxil®), sertraline (Zoloft®), or trazodone (Desyrel®); anxiolytics comprising lorazepam (Ativan®) or oxazepam (Serax®); antipsychotic comprising aripiprazole (Abilify®), clozapine (Clozaril®), haloperidol (Haldol®), olanzapine (Zyprexa®), quetiapine (Seroquel®), risperidone (Risperdal®), or ziprasidone (Geodon®); tricyclic antidepressants comprising am itriptyline, amoxapine, desipramine (Norpramin®), doxepin, imipramine (Tofranil®), nortriptyline (Pamelor®), protriptyline, trimipramine; benzodiazepines comprising lorazepam, oxazepam or temazepam; sleeping treatments comprising zolpidem (Ambien®), zaleplon (Sonata®), eszopiclone (Lunesta®), phenobarbital, or chloral hydrate; atypical antipsychotics comprising risperidone, olanzapine, or quetiapine; classical antipsychotics comprising haloperidol; non-steroidal antiinflammatory drugs (NSAIDs, ibuprofen, naproxen, diclofenac, acetylsalicylic acid), acetaminophen, or alternative treatments or dietary supplements comprising amino acids (alanine, aspartate, glutamate, etc.), α-ketoglutarate, pyridoxal phosphate, vitamins (retinol (A), thiamine (B1), riboflavin (B2), niacinamide (B3), adenine (B4), pantothenic acid (B5), pyridoxine (B6), biotin (B7), adenylate (B8), carnitine (BT), folic acid (B9), cobalamin (B12), ascorbic acid (C), cholecalciferol (D), tocopherol (E), essential fatty acids (F), catechol (J), phylloquinone (K), salicylic acid (S), S-methylmethionine (U), inositol, choline), huperzine A, tramiprosate, caprylic acid, coconut oil, omega-3 fatty acids (fish oil, Lovaza®, Vascepa®, Epanova®, Omtryg®, Vscazen®), coenzyme Q10, phosphatidylserine, coral calcium, or Ginkgo biloba extracts.

In another embodiment described herein, the subject or population of subjects has liver disease. In one aspect, the subject or population of subjects has decreased liver function. In another aspect, the control sample is from a subject or population of subjects with normal cognition. In another aspect, the control sample is from a subject or population of subjects not having AD or poor cognition. In another aspect, the control sample is from a subject or population of subjects not having liver disease. In another aspect, the sample comprises whole blood, serum, plasma, or cerebral spinal fluid (CSF). In another aspect, the sample comprises blood. In another aspect, the sample comprises cerebral spinal fluid (CSF).

Another embodiment described herein is the use of one or more biomarkers selected from alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, or a combination thereof and genetic analysis to identify mutant, variant, or isozyme ALT, AST, or related genes for identifying or diagnosing Alzheimer's Disease (AD) or poor cognitive performance in a subject or population of subjects.

Another embodiment described herein is the use of one or more biomarkers selected from alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, or a combination thereof and genetic analysis to identify mutant, variant, or isozyme ALT, AST, or related genes for stratifying a population of subjects for determining risk of developing or having Alzheimer's Disease (AD) or poor cognitive performance.

Another embodiment described herein is the use of one or more biomarkers selected from alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, or a combination thereof and genetic analysis to identify mutant, variant, or isozyme ALT, AST, or related genes for determining the risk of a subject or population of subjects developing Alzheimer's Disease (AD) or poor cognitive performance.

Another embodiment described herein is the use of one or more biomarkers selected from alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, or a combination thereof and genetic analysis to identify mutant, variant, or isozyme ALT, AST, or related genes for identifying or diagnosing Alzheimer's Disease (AD) or poor cognitive performance in a subject or population of subjects; and administering a treatment to the subject determined to have AD or poor cognitive performance.

Another embodiment described herein is a use as described herein wherein the subject or population of subjects are further evaluated using clinical assays, magnetic resonance imaging (MRI), or position emission tomography (PET) for one or more of cerebral spinal fluid (CSF) amyloid-β1-42 levels; amyloid-β deposition; CSF phosphorylated tau levels; CSF total tau levels; brain glucose metabolism; brain atrophy, or a combination thereof. In one aspect, the subject or population of subjects diagnosed as having or at risk for AD or poor cognitive performance has one or more of: lower cerebral spinal fluid (CSF) amyloid-β1-42 levels; increased amyloid-β deposition; greater CSF phosphorylated tau levels; greater CSF total tau levels; reduced brain glucose metabolism; greater brain atrophy; or a combination thereof.

Another embodiment described herein is the use further comprising the administration of a treatment to the subject(s) determined to have a risk of developing or having AD or poor cognitive performance.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All the various embodiments, aspects, and options disclosed herein can be combined in any variations or iterations. The scope of the compositions, formulations, methods, and processes described herein include all actual or potential combinations of embodiments, aspects, options, examples, and preferences herein described. The compositions, formulations, or methods described herein may omit any component or step, substitute any component or step disclosed herein, or include any component or step disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the specification discloses and describes merely exemplary embodiments. All patents and publications cited herein are incorporated by reference herein for the specific teachings thereof.

### REFERENCES

1. Toledo et al., “Alzheimer's Disease Neuroimaging Initiative and the Alzheimer Disease Metabolomics Consortium. Metabolic network failures in Alzheimer's disease: a biochemical road map,” Alzheimers Dement. 13(9): 965-984 (2017).
2. Clarke et al., “Metabolic dysfunction in Alzheimer's disease: from basic neurobiology to clinical approaches,” J. Alzheimers Dis. 64(s1): S405-S426 (2018).
3. Kapogiannis D, Mattson M P. Disrupted energy metabolism and neuronal circuit dysfunction in cognitive impairment and Alzheimer's disease. Lancet Neurol. 10(2): 187-198 (2011).
4. Craft S., “The role of metabolic disorders in Alzheimer disease and vascular dementia: two roads converged,” Arch Neurol. 66(3): 300-305 (2009).
5. Sookoian et al., “Serum aminotransferases in nonalcoholic fatty liver disease are a signature of liver metabolic perturbations at the amino acid and Krebs cycle level,” Am. J. Clin. Nutr. 103(2): 422-434 (2016).
6. Sookoian and Pirola, “Alanine and aspartate aminotransferase and glutamine-cycling pathway: their roles in pathogenesis of metabolic syndrome,” World J. Gastroenterol. 18(29): 3775-3781 (2012).
7. Goessling et al. “Aminotransferase levels and 20-year risk of metabolic syndrome, diabetes, and cardiovascular disease,” Gastroenterology 135(6): 1935-1944 (2008).
8. Sattar et al., “West of Scotland Coronary Prevention Study. Elevated alanine aminotransferase predicts new-onset type 2 diabetes independently of classical risk factors, metabolic syndrome, and C-reactive protein in the West of Scotland Coronary Prevention Study,” Diabetes 53(11): 2855-2860 (2004).
9. Santos et al., “Pathophysiologic relationship between Alzheimer's disease, cerebrovascular disease, and cardiovascular risk: a review and synthesis,” Alzheimers Dement (Amst). 7: 69-87 (2017).
10. Fillit et al., “Cardiovascular risk factors and dementia,” Am. J. Geriatr. Pharmacother. 6(2): 100-118 (2008).
11. Jack et al., “NIA-AA research framework: toward a biological definition of Alzheimer's disease,” Alzheimers Dement. 14(4): 535-562 (2018).
12. Alzheimer's Disease Neuroimaging Initiative (ADNI) website. adni.loni.usc.edu/. Accessed Jul. 8, 2019.
13. Saykin et al., “Alzheimer's Disease Neuroimaging Initiative. Genetic studies of quantitative MCI and AD phenotypes in ADNI: progress, opportunities, and plans,” Alzheimers Dement. 11(7): 792-814 (2015).
14. Weiner et al., “Alzheimer's Disease Neuroimaging Initiative. Recent publications from the Alzheimer's Disease Neuroimaging Initiative: Reviewing progress toward improved AD clinical trials,” Alzheimers Dement. 13(4): e1-e85 (2017).
15. Petersen et al., “Alzheimer's Disease Neuroimaging Initiative (ADNI): clinical characterization,” Neurology 74(3): 201-209 (2010).
16. Rattanabannakit et al., “The Cognitive Change Index as a measure of self and informant perception of cognitive decline: relation to neuropsychological tests,” J. Alzheimers Dis. 51(4): 1145-1155 (2016).
17. McKhann et al., “Clinical diagnosis of Alzheimer's disease: report of the NINCDS-ADRDA Work Group under the auspices of Department of Health and Human Services Task Force on Alzheimer's Disease,” Neurology 34(7): 939-944 (1984).
18. Aisen et al., “Alzheimer's Disease Neuroimaging Initiative. Clinical core of the Alzheimer's Disease Neuroimaging Initiative: progress and plans,” Alzheimers Dement. 6(3): 239-246 (2010).
19. Crane et al., “Alzheimer's Disease Neuroimaging Initiative. Development and assessment of a composite score for memory in the Alzheimer's Disease Neuroimaging Initiative (ADNI),” Brain Imaging Behav. 6(4): 502-516 (2012).
20. Gibbons et al., “Alzheimer's Disease Neuroimaging Initiative. A composite score for executive functioning, validated in Alzheimer's Disease Neuroimaging Initiative (ADNI) participants with baseline mild cognitive impairment,” Brain Imaging Behav. 6(4): 517-527 (2012).
21. Jack et al., “Alzheimer's Disease Neuroimaging Initiative. Update on the magnetic resonance imaging core of the Alzheimer's disease neuroimaging initiative,” Alzheimers Dement. 6(3): 212-220 (2010).
22. Jack et al., “The Alzheimer's Disease Neuroimaging Initiative (ADNI): MRI methods,” J. Magn. Reson. Imaging. 27(4): 685-691 (2008).
23. Kim et al., “Alzheimer's Disease Neuroimaging Initiative (ADNI). Influence of genetic variation on plasma protein levels in older adults using a multi-analyte panel,” PLoS One 8(7): e70269 (2013).
24. Nho et al., “Multi-Institutional Research on Alzheimer Genetic Epidemiology (MIRAGE) Study; AddNeuroMed Consortium; Indiana Memory and Aging Study; Alzheimer's Disease Neuroimaging Initiative (ADNI). Whole-exome sequencing and imaging genetics identify functional variants for rate of change in hippocampal volume in mild cognitive impairment,” Mol, Psychiatry 18(7): 781-787 (2013).
25. Nho et al., “MIRAGE (Multi-Institutional Research on Alzheimer Genetic Epidemiology) Study; AddNeuroMed Consortium; Indiana Memory and Aging Study; Alzheimer's Disease Neuroimaging Initiative. Protective variant for hippocampal atrophy identified by whole exome sequencing,” Ann. Neurol. 77(3): 547-552 (2015).
26. Fischl et al., “Cortical surface-based analysis, II: inflation, flattening, and a surface-based coordinate system,” Neuroimage 9(2): 195-207 (1999).
27. Dale et al., “Cortical surface-based analysis, I: segmentation and surface reconstruction,” Neuroimage 9(2): 179-194 (1999).
28. Chung et al., “General multivariate linear modeling of surface shapes using SurfStat,” Neuroimage 53(2): 491-505 (2010).
29. Risacher et al., “Alzheimer's Disease Neuroimaging Initiative (ADNI). APOE effect on Alzheimer's disease biomarkers in older adults with significant memory concern,” Alzheimers Dement. 11(12): 1417-1429 (2015).
30. Bittner et al., “Technical performance of a novel, fully automated electrochemiluminescence immunoassay for the quantitation of β-amyloid (1-42) in human cerebrospinal fluid,” Alzheimers Dement. 12(5): 517-526 (2016).
31. Hansson et al., “Swedish BioFINDER study group; Alzheimer's Disease Neuroimaging Initiative. CSF biomarkers of Alzheimer's disease concord with amyloid-β PET and predict clinical progression: a study of fully automated immunoassays in BioFINDER and ADNI cohorts,” Alzheimers Dement. 14(11): 1470-1481 (2018).
32. Noble, et al., “Hippocampal volume varies with educational attainment across the life-span,” Front Hum Neurosci. 6: 307 (2012).
33. Worsley K J. SurfStat. www.math.mcgill.ca/keith/surfstat/. Accessed Jul. 8, 2019.
34. SPM: Statistical Parametric Mapping. www.fil.ion.ucl.ac.uk/spm/. Accessed Jul. 8, 2019.
35. Hagler et al., “Smoothing and cluster thresholding for cortical surface-based group analysis of fMRI data,” Neuroimage 33(4): 1093-1103 (2006).
36. Hayasaka et al., “Nonstationary cluster-size inference with random field and permutation methods,” Neuroimage 22(2): 676-687 (2004).
37. Worsley et al., “Unified univariate and multivariate random field theory,” Neuroimage 23(suppl 1): S189-S195 (2004).
38. Benjamini and Hochberg, “Controlling the false discovery rate: a practical and powerful approach to multiple testing,” J. R. Stat. Soc. Series B Stat. Methdol. 57(1): 289-300 (1995).
39. Jack et al., “NIA-AA research framework: towards a biological definition of Alzheimer's disease,” presented at: Alzheimer's Association International Conference; London, England Nov. 27, 2017.
40. Giambattistelli et al., “Effects of hemochromatosis and transferrin gene mutations on iron dyshomeostasis, liver dysfunction and on the risk of Alzheimer's disease,” Neurobiol. Aging 33(8): 1633-1641 (2012).
41. Pietzner et al., “Hepatic steatosis is associated with adverse molecular signatures in subjects without diabetes,” J. Clin. Endocrinol. Metab. 103(10): 3856-3868 (2018).
42. Varma et al., “Brain and blood metabolite signatures of pathology and progression in Alzheimer disease: a targeted metabolomics study,” PLoS Med. 15(1): e1002482 (2018).
43. Kaddurah-Daouk et al., “Alterations in metabolic pathways and networks in mild cognitive impairment and early Alzheimer's disease,” Alzheimers Dement. 9(4)(suppl): P571 (2013).
44. Kaddurah-Daouk et al., “Metabolomic changes in autopsy-confirmed Alzheimer's disease,” Alzheimers Dement. 7(3): 309-317 (2011).
45. Botros and Sikaris, “The de ritis ratio: the test of time.” Clin. Biochem. Rev. 34(3): 117-130 (2013).
46. Mosconi et al., “Hypometabolism exceeds atrophy in presymptomatic early-onset familial Alzheimer's disease,” J. Nucl. Med. 47(11): 1778-1786 (2006).
47. Drzezga et al., “Cerebral metabolic changes accompanying conversion of mild cognitive impairment into Alzheimer's disease: a PET follow-up study,” Eur. J. Nucl. Med. Mol. Imaging 30(8): 1104-1113 (2003).
48. Rui L. Energy metabolism in the liver,” Compr. Physiol. 4(1): 177-197 (2014).
49. Ellinger et al., “Role of aminotransferases in glutamate metabolism of human erythrocytes,” J. Biomol. NMR 49(3-4):221-229 (2011).
50. Qian et al., “Hepatic ALT isoenzymes are elevated in gluconeogenic conditions including diabetes and suppressed by insulin at the protein level,” Diabetes Metab. Res. Rev. 31(6): 562-571 (2015).
51. Reis et al., “Neuro-transmitters in the central nervous system & their implication in learning and memory processes,” Curr. Med. Chem. 16(7): 796-840 (2009).
52. Fleck et al., “Aspartate and glutamate mediate excitatory synaptic transmission in area CA1 of the hippocampus,” J. Neurosci. 13(9): 3944-3955 (1993).
53. Francis P T, “Glutamatergic systems in Alzheimer's disease,” Int J. Geriatr. Psychiatry 18(suppl 1): S15-S21 (2003).
54. Kamada et al., “Impact of plasma transaminase levels on the peripheral blood glutamate levels and memory functions in healthy subjects,” BBA Clin. 5: 101-107 (2016).
55. Alfredsson et al., “Relationships between glutamate and monoamine metabolites in cerebrospinal fluid and serum in healthy volunteers,” Biol. Psychiatry 23(7): 689-697 (1988).
56. Wang et al., “Plasma metabolite profiles of Alzheimer's disease and mild cognitive impairment,” J. Proteome Res. 13(5): 2649-2658 (2014).
57. Lowe et al., “Ante mortem cerebral amino acid concentrations indicate selective degeneration of glutamate-enriched neurons in Alzheimer's disease,” Neuroscience 38(3): 571-577 (1990).
58. Fayed et al., “Brain glutamate levels are decreased in Alzheimer's disease: a magnetic resonance spectroscopy study,” Am. J. Alzheimers Dis. Other Demen. 26(6): 450-456 (2011).
59. Procter et al., “Evidence of glutamatergic denervation and possible abnormal metabolism in Alzheimer's disease,” J. Neurochem. 50(3): 790-802 (1988).
60. Liu et al., “Complex association between alanine aminotransferase activity and mortality in general population: a systematic review and meta-analysis of prospective studies,” PLoS One 9(3): e91410 (2014).
61. Peltz-Sinvani et al., “Low ALT levels independently associated with 22-year all-cause mortality among coronary heart disease patients,” J. Gen. Intern. Med. 31(2): 209-214 (2016).
62. Vespasiani-Gentilucci et al., “Low alanine aminotransferase levels in the elderly population: frailty, disability, sarcopenia, and reduced survival,” J. Gerontol A Biol. Sci. Med. Sci. 73(7): 925-930 (2018).
63. Elinav al., “Correlation between serum alanine aminotransferase activity and age: an inverted U curve pattern,” Am. J. Gastroenterol. 100(10): 2201-2204 (2005).
64. Kaiser et al., “Age-related glutamate and glutamine concentration changes in normal human brain: ¹H MR spectroscopy study at 4 T,” Neurobiol Aging. 26(5): 665-672 (2005).
65. Guerreiro and Bras, “The age factor in Alzheimer's disease,” Genome Med. 7: 106 (2015).
66. Katsiki et al., “Is nonalcoholic fatty liver disease indeed the hepatic manifestation of metabolic syndrome?” Curr. Vasc. Pharmacol. 16(3): 219-227 (2018).
67. Weinstein et al., “Association of nonalcoholic fatty liver disease with lower brain volume in healthy middle-aged adults in the Framingham Study,” JAMA Neurol. 75(1): 97-104 (2018).
68. Bedogni et al., “Relationship between glucose metabolism and non-alcoholic fatty liver disease severity in morbidly obese women,” J. Endocrinol. Invest. 37(8): 739-744 (2014).
69. Perla et al., “The role of lipid and lipoprotein metabolism in non-alcoholic fatty liver disease,” Children (Basel) 4(6): E46 (2017).
70. Kellett et al., “Plasma alkaline phosphatase is elevated in Alzheimer's disease and inversely correlates with cognitive function,” Int. J. Mol. Epidemiol. Genet. 2(2): 114-121 (2011).
71. Moss D W. “Physicochemical and pathophysiological factors in the release of membrane-bound alkaline phosphatase from cells,” Clin. Chim. Acta 257(1): 133-140 (1997).
72. Goldstein et al., “Expression of alkaline phosphatase loci in mammalian tissues,” Proc. Natl. Acad. Sci. USA 77(5): 2857-2860 (1980).
73. Fonta et al. “Areal and subcellular localization of the ubiquitous alkaline phosphatase in the primate cerebral cortex: evidence for a role in neurotransmission,” Cereb. Cortex 14(6): 595-609 (2004).
74. Waymire et al., “Mice lacking tissue non-specific alkaline phosphatase die from seizures due to defective metabolism of vitamin B-6,” Nat. Genet. 11(1): 45-51 (1995).
75. Narisawa et al., “Abnormal vitamin B6 metabolism in alkaline phosphatase knock-out mice causes multiple abnormalities, but not the impaired bone mineralization,” J. Pathol. 193(1): 125-133 (2001).
76. Langer et al., “The ectonucleotidases alkaline phosphatase and nucleoside triphosphate diphosphohydrolase 2 are associated with subsets of progenitor cell populations in the mouse embryonic, postnatal and adult neurogenic zones,” Neuroscience 150(4): 863-879 (2007).
77. Yamashita et al., “Measurement of serum alkaline phosphatase isozyme I in brain-damaged patients,” Neurol. Med Chir. (Tokyo) 29(11): 995-998 (1989).
78. Gjerde et al., “Serum gamma-glutamyltransferase: an epidemiological indicator of alcohol consumption?” Br. J. Addict. 82(9): 1027-1031 (1987).
79. Nho et al., “Association of Altered Liver Enzymes With Alzheimer Disease Diagnosis, Cognition, Neuroimaging Measures, and Cerebrospinal Fluid Biomarkers,” JAMA Netw Open 2(7):e197978 (2019).

### EXAMPLES

### Example 1

### Study Population

Individuals in this study were participants of ADNI. The initial phase (ADNI-1) was launched in 2003 to test whether serial MRI markers, PET markers, other biological markers, and clinical and neuropsychological assessment could be combined to measure the progression of mild cognitive impairment (MCI) and early AD. The initial phase was extended to subsequent phases (ADNI-GO, ADNI-2, and ADNI-3) for follow-up of existing participants and additional new enrollments. Inclusion and exclusion criteria, clinical and neuroimaging protocols, and other information are reported elsewhere [12-14]. Demographic and clinical information, raw data from neuroimaging scans, CSF biomarkers, information on APOE status, and cognitive scores were downloaded from the ADNI data repository [12]. Baseline data were collected from Sep. 1, 2005, to Aug. 31, 2013. Written informed consent was obtained at enrollment, which included permission for analysis and data sharing. This study was approved by each participating site's institutional review board. This report followed the Strengthening the Reporting of Observational Studies in Epidemiology (STROBE) reporting guidelines for cohort studies.

### Liver Function Markers

Five laboratory tests were downloaded from the ADNI data repository and used in the study: total bilirubin, albumin, alkaline phosphatase, ALT, and AST. The liver function markers followed a normal distribution after log transformation. For each marker, participants with values greater or smaller than 4 SDs from its mean value were considered outliers and were removed. To determine if outliers had a significant effect on the findings a sensitivity analysis was performed and observed few differences (or slightly more significant), if any, in results when including outliers (Table 1).

TABLE 1
Sensitivity Analysis for Alzheimer Disease Diagnosis (CN vs. AD)
Group Differences in Liver Function Biomarkers

| | Excluding Outliers | | | Including Outliers | | |
|---|---|---|---|---|---|---|
| Liver Function | Odds | | corrected | Odds | | Corrected |
| Marker | Ratio | 95% CI | P Value | Ratio | 95% CI | P Value |
| Albumin | 5.789 | 0.040, 843.993 | 4.90 × 10⁻¹ | 5.789 | 0.040, 843.993 | 4.90 × 10⁻¹ |
| Alkaline | 3.620 | 0.844, 15.529 | 1.25 × 10⁻¹ | 3.620 | 0.844, 15.529 | 1.25 × 10⁻¹ |
| Phosphatase | | | | | | |
| ALT | 0.133 | 0.042, 0.422 | 3.63 × 10⁻³ | 0.120 | 0.039, 0.374 | 2.55 × 10⁻³ |
| AST | 0.229 | 0.045, 1.175 | 1.25 × 10⁻¹ | 0.203 | 0.041, 1.021 | 1.06 × 10⁻¹ |
| AST:ALT | 7.932 | 1.673, 37.617 | 2.73 × 10⁻² | 9.815 | 2.156, 44.670 | 9.41 × 10⁻³ |
| Total Bilirubin | 1.405 | 0.585, 3.377 | 4.90 × 10⁻¹ | 1.405 | 0.585, 3.377 | 4.90 × 10⁻¹ |

Age, sex, body mass index, and APOE ε4 status included as covariates.
Abbreviations: AST: Aspartate aminotransferase; ALT: alanine aminotransferase.

### Dementia Diagnosis

Participants in ADNI were classified as cognitively normal controls (CN) or having significant memory concerns (SMC), MCI, or mild clinical AD. Criteria for classification were as follows: Mini-Mental State Examination score range (range, 0 [worst] to 30 [best]) for CN and MCI was 24 to 30, and for AD was 20 to 26; and overall Clinical Dementia Rating score (range for each, 0 [best] to 3 [worst]) for CN was 0, for MCI was 0.5 with a mandatory requirement of memory box score of 0.5 or greater, and for AD was 0.5 or 1 [15]. Cognitively normal controls did not have any significant impairment in cognition or activities of daily living. Participants with SMC had normal cognition and no significant impairment in activities of daily living, but had a score of 16 or more on the first 12 items of the self-report version of the Cognitive Change Index (range, 12 [no change] to 60 [severe change]) [16]. Participants with MCI had cognitive impairments in memory and/or other domains but were able to perform activities of daily living and did not qualify for a diagnosis of dementia [15]. Participants with AD had to meet the National Institute of Neurological and Communicative Disorders and Stroke—AD and Related Disorders Association criteria for probable AD [17]. Participants from the ADNI-1 cohort with MCI were all classified as late MCI, with a memory impairment approximately 1.5 SD below education-adjusted norms. In the ADNI-GO and ADNI-2 cohort, participants with MCI were classified as either early MCI, with a memory impairment approximately 1 SD below education-adjusted norms, or late MCI (same criteria as in ADNI-1). Both ADNI-1 and ADNI-GO and ADNI-2 participants met the criteria for amnestic MCI, but many in the ADNI-GO and ADNI-2 cohort included the earlier stage MCI designation (i.e., early MCI) [18].

Cognition Composite scores were used to measure memory and executive functioning. A memory composite score was created from the following: memory tasks from the Alzheimer Disease Assessment Scale—cognitive subscale, the Rey Auditory Verbal Learning Test, memory components of the Mini-Mental State Examination, and the Logical Memory task [19]. An executive function composite score included the following: Wechsler Adult Intelligence Scale-Revised Digit Symbol Substitution task and Digit Span backward task, Trail Making Test Parts A and B, category fluency (animals and vegetables), and 5 clock drawing items. Composite scores have a mean of 0 and an SD of 1 [20].

### MRI Scans

Baseline T1-weighted brain MRI scans were acquired using a sagittal 3-dimensional magnetization prepared rapid gradient echo scans following the ADNI MRI protocol [21, 22]. As previously detailed, FreeSurfer, version 5.1, a widely used automated MRI analysis approach, was used to process MRI scans and extract whole-brain and region-of-interest (ROI)-based neuroimaging endophenotypes including volumes and cortical thickness determined by automated segmentation and parcellation [23-25]. The cortical surface was reconstructed to measure thickness at each vertex. The cortical thickness was calculated by taking the Euclidean distance between the gray and white boundary and the gray and CSF boundary at each vertex on the surface [26-28].

### PET Scans

Preprocessed fludeoxyglucose (FDG), fluorine 18 (¹⁸F), and [¹⁸F] florbetapir PET scans (coregistered, averaged, standardized image and voxel size, and uniform resolution) were downloaded from the ADNI Laboratory of Neuro Imaging (LONI) site [12] as described in previously reported methods for acquisition and processing of PET scans [23, 29]. For [¹⁸F] FDG-PET, scans were intensity normalized using a pons ROI to create [¹⁸F] FDG standardized uptake value ratio (SUVR) images. For [¹⁸F] florbetapir PET, scans were intensity normalized using a whole cerebellum reference region to create SUVR images.

### CSF Biomarkers

The ADNI generated CSF biomarkers (amyloid-β 1-42, total tau [t-tau], and phosphorylated tau₁₈₁, [p-tau₁₈₁]) in pristine aliquots of 2401 ADNI CSF samples using the validated and highly automated Roche Elecsys® electrochemiluminescence immunoassays [30, 31] and the same reagent lot for each of these 3 biomarkers. Cerebrospinal fluid biomarker data were downloaded from the ADNI LONI site [12].

### Statistical Analysis

Statistical analysis was conducted from Nov. 1, 2017, to Feb. 28, 2019. Logistic regression analysis was performed to explore the diagnostic group differences between AD diagnosis and each liver function marker separately. Age, sex, body mass index (BMI), and APOE ε4 status were used as covariates. A linear regression analysis was performed to access the association of liver function markers with composite scores for memory and executive functioning using age, sex, years of education, BMI, and APOE ε4 status as covariates. A linear regression was also performed analysis using age, sex, BMI, and APOE ε4 status as covariates.

### ROI-Based Analysis of Structural MRI and PET Scans

Mean hippocampal volume was used as an MRI-related phenotype. For FDG-PET, a mean standardized uptake value ratio (SUVR) value was extracted from a global cortical ROI representing regions where patients with AD show decreased glucose metabolism relative to CN participants from the full ADNI-1 cohort, normalized to pons [29]. For [¹⁸F] florbetapir PET, a mean SUVR value was extracted using MarsBaR from a global cortical region generated from an independent comparison of ADNI-1 [¹¹C] Pittsburgh Compound B SUVR scans (regions where AD>CN). A linear regression analysis was performed using age, sex, BMI, and APOE ε4 status as covariates to evaluate the association of liver function markers with AD-related endophenotypes from MRI and PET scans. For hippocampal volume, years of education, intracranial volume, and magnetic field strength were added as additional covariates [32].

### Whole-Brain Imaging Analysis

The SurfStat software package [33] was used to perform a multivariable analysis of cortical thickness to examine the association of liver function markers with brain structural changes on a vertex-by-vertex basis using a general linear model approach [28]. General linear models were developed using age, sex, years of education, intracranial volume, BMI, APOE ε4 status, and magnetic field strength as covariates. The processed FDG-PET and [¹⁸F] florbetapir PET images were used to perform a voxelwise statistical analysis of the association of liver function markers with brain glucose metabolism and amyloid-β accumulation across the whole brain using SPM8 [34]. A multivariable regression analysis was performed using age, sex, BMI, and APOE ε4 status as covariates. In the whole-brain surface-based analysis, the adjustment for multiple comparisons was performed using the random field theory correction method with P<0.05 adjusted as the level for significance [35-37]. In the voxelwise whole-brain analysis, the significant statistical parameters were selected to correspond to a threshold of P<0.05 (false discovery rate [FDR]—corrected) [38].

### Multiple Testing Correction

Results of the analysis of liver function markers with AD diagnosis groups, cognitive composite measures, and A/T/N biomarkers for AD separately were corrected for multiple testing using the FDR with the Benjamini-Hochberg procedure (p.adjust command in R, R Project for Statistical Computing).

### Example 2

### Study Sample

These analyses included 1581 ADNI participants (407 CN, 20 with SMC, 298 with early MCI, 544 with late MCI, and 312 with AD). Demographic information as well as mean and SD of liver function markers stratified by clinical diagnosis are presented in Table 2.

TABLE 2
Demographic Information of ADNI Participants

| | CN | SMC | EMCI | LMCI | AD | |
|---|---|---|---|---|---|---|
| Variable | (N = 407) | (N = 20) | (N = 298) | (N = 544) | (N = 312) | P Value |
| Age (years) | 74.5 (5.7) | 71.6 (5.1) | 70.9 (7.4) | 73.5 (7.6) | 74.6 (7.7) | 1.94 × 10⁻¹¹ |
| Sex: (Male/Female) | 206/201 | 10/10 | 166/132 | 331/213 | 171/141 | 3.35 × 10⁻² |
| Education (years) | 16.3 (2.7) | 16.8 (2.2) | 16.0 (2.6) | 15.9 (3.0) | 15.1 (3.0) | 1.59 × 10⁻⁶ |
| BMI (kg/m²) | 27.4 (4.5) | 29.3 (4.6) | 28.4 (5.3) | 26.9 (4.3) | 26.1 (4.4) | 6.94 × 10⁻⁹ |
| APOE ε4 status (0/1) | 294/113 | 15/5 | 169/129 | 249/295 | 98/214 | <2.20 × 10⁻¹⁶ |
| Albumin (g/dL) | 4.09 (0.31) | 3.94 (0.25) | 4.10 (0.29) | 4.14 (0.32) | 4.11 (0.32) | 1.29 × 10⁻² |
| Alkaline | 75.3 (20.7) | 74.4 (14.1) | 71.1 (17.9) | 74.9 (22.4) | 76.2 (19.2) | 2.09 × 10⁻² |
| phosphatase (U/L) | | | | | | |
| ALT (U/L) | 21.5 (8.4) | 24.2 (6.6) | 22.2 (9.3) | 20.7 (8.0) | 19.5 (7.0) | 5.44 × 10⁻⁵ |
| AST (U/L) | 24.3 (6.2) | 25.2 (8.0) | 24.7 (7.4) | 24.1 (6.1) | 23.7 (6.3) | 4.66 × 10⁻¹ |
| AST:ALT | 1.22 (0.32) | 1.06 (0.22) | 1.18 (0.31) | 1.25 (0.31) | 1.29 (0.35) | 1.22 × 10⁻⁵ |
| Total bilirubin (mg/dL) | 0.52 (0.26) | 0.44 (0.14) | 0.54 (0.28) | 0.53 (0.24) | 0.56 (0.30) | 3.02 × 10⁻¹ |
| Memory composite | 0.94 (0.53) | 0.99 (0.57) | 0.53 (0.49) | −0.04 (0.57) | −0.77 (0.54) | <2.20 × 10⁻¹⁶ |
| score | | | | | | |
| Executive function | 0.75 (0.71) | 0.82 (0.64) | 0.47 (0.76) | 0.01 (0.80) | −0.88 (0.82) | <2.20 × 10⁻¹⁶ |
| composite score | | | | | | |

Abbreviations: AD: Alzheimer's Disease; ALT: alanine aminotransferase; AST: Aspartate aminotransferase; BMI: Body Mass Index; CN: Cognitively Normal; EMCI: early mild cognitive impairment; LMCI: late MCI; SMC: subjective memory concerns.

### Diagnostic Group Difference of Liver Function Markers With AD Diagnosis

Levels of ALT were significantly decreased in AD compared with CN (odds ratio, 0.133; 95% Cl, 0.042-0.422; P=0.004) (Table 3), while AST to ALT ratio values were significantly increased in AD (odds ratio, 7.932; 95% CI, 1.673-37.617; P=0.03). There was a trend to suggest that ALT levels were increased and AST to ALT ratio values were decreased in MCI compared with CN, but these became nonsignificant after adjustment for multiple comparisons (Table 4).

TABLE 3
Results of Association of Liver Function Biomarkers
with Alzheimer Disease Diagnosis^{†}

| | Odds Ratio | | Corrected |
|---|---|---|---|
| Liver Function Marker | (95% Cl) | | P Value |
| Albumin, g/dL | 5.789 | (0.040-843.993) | 0.49 |
| Alkaline phosphatase, U/L | 3.620 | (0.844-15.529) | 0.12 |
| ALT, U/L | 0.133 | (0.042-0.422) | 0.004 |
| AST, U/L | 0.229 | (0.045-1.175) | 0.12 |
| AST to ALT ratio | 7.932 | (1.673-37.617) | 0.03 |
| Total bilirubin, mg/dL | 1.405 | (0.585-3.377) | 0.49 |

^{†}Cognitively Normal vs. Alzheimer Disease. Analyses were adjusted for age, sex, body mass index, and APOE ε4 status. Abbreviations: ALT, alanine aminotransferase; AST, aspartate aminotransferase.

TABLE 4
Diagnostic Group Differences in Liver Function Biomarkers

| | CN vs. EMCI | | CN vs. LMCI | | CN vs. AD | |
|---|---|---|---|---|---|---|
| | Odds | | Odds | | Odds | |
| Liver Function Marker | Ratio | 95% CI | Ratio | 95% CI | Ratio | 95% CI |
| Albumin (g/dL) | 0.811 | 0.006, 117.8 | 19.623 | 0.333, 1156.4 | 5.789 | 0.040, 844.0 |
| Alkaline Phosphatase (U/L) | 0.144 | 0.035, 0.588 | 0.883 | 0.290, 2.694 | 3.620 | 0.844, 15.53 |
| ALT (U/L) | 0.561 | 0.196, 1.604 | 0.291 | 0.112, 0.755 | 0.133 | 0.042, 0.422 |
| AST (U/L) | 0.742 | 0.170, 3.230 | 0.393 | 0.102, 1.506 | 0.229 | 0.045, 1.175 |
| AST:ALT | 1.240 | 0.278, 5.540 | 3.783 | 0.995, 14.38 | 7.932 | 1.673, 37.62 |
| Total Bilirubin (mg/dL) | 1.319 | 0.556, 3.133 | 0.887 | 0.417, 1.886 | 1.405 | 0.585, 3.377 |

Age, sex, body mass index, and APOE ε4 status were included as covariates.
Abbreviations: AD: Alzheimer's Disease; ALT: alanine aminotransferase; AST: Aspartate aminotransferase; BMI: Body Mass Index; CN: Cognitively Normal; EMCI: early mild cognitive impairment; LMCI: late MCI.

### Cognition

After adjusting for multiple comparison correction using FDR, significant associations of liver function markers with cognition were identified (Table 5). Higher levels of alkaline phosphatase and AST to ALT ratio were associated with lower memory scores (alkaline phosphatase: β[SE], −0.416 [0.162]; P=0.02; AST to ALT ratio: β[SE], −0.465 [0.180]; P=0.02) and executive functioning scores (alkaline phosphatase: β[SE], −0.595 [0.193]; P=0.006; AST to ALT ratio: β[SE], −0.679 [0.215]; P=0.006). Higher ALT levels were associated with higher memory scores (β[SE], 0.397 [0.128]; P=0.006) and executive functioning scores (β[SE], 0.637 [0.152]; P<0.001), whereas higher AST levels were associated with higher executive functioning scores (β[SE], 0.607 [0.215]; P=0.01).

TABLE 5
Results of Association of Liver Function Biomarkers With
Composite Cognitive Performance Measures^{†}

| | Memory | | | Executive Function | | |
|---|---|---|---|---|---|---|
| | Composite Score | | | Composite Score | | |
| | | | Corrected | | | Corrected |
| Liver Function Marker | β (SE) | | P Value | β (SE) | | P Value |
| Albumin, g/dL | −0.872 | (0.576) | 0.17 | −0.203 | (0.689) | 0.77 |
| Alkaline phosphatase, U/L | −0.416 | (0.162) | 0.02 | −0.595 | (0.193) | 0.006 |
| ALT, U/L | 0.397 | (0.128) | 0.006 | 0.637 | (0.152) | <0.001 |
| AST, U/L | 0.339 | (0.180) | 0.09 | 0.607 | (0.215) | 0.01 |
| AST to ALT ratio | −0.465 | (0.180) | 0.02 | −0.679 | (0.215) | 0.006 |
| Total bilirubin, mg/dL | −0.068 | (0.103) | 0.61 | −0.066 | (0.123) | 0.65 |

^{†}Analyses were adjusted for age, sex, educational level, body mass index, and APOE ε4 status. Abbreviations: ALT, alanine aminotransferase; AST, aspartate aminotransferase.

### Biomarkers of Amyloid-β

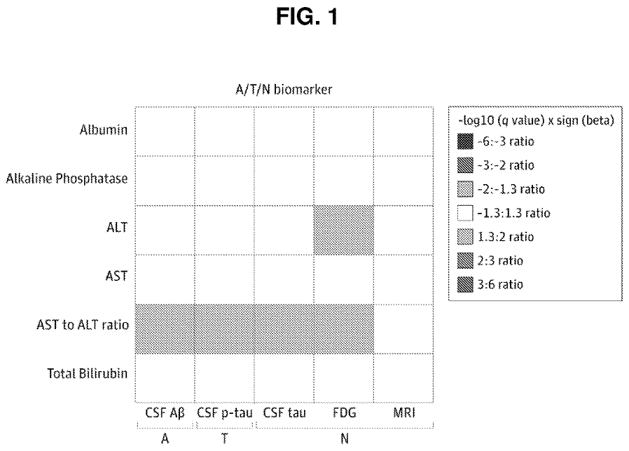

CSF amyloid-β 1-42 levels and a global cortical amyloid deposition measured from amyloid PET scans were used as biomarkers of amyloid-β. The regression coefficient of the AST to ALT ratio showed a negative association with CSF amyloid-β 1-42 levels (β[SE], −0.170 [0.061]; P=0.04), indicating that higher AST to ALT ratio values were associated with CSF amyloid-β 1-42 positivity (FIG. 1). However, there was no significant correlation between liver function markers and global cortical amyloid deposition.

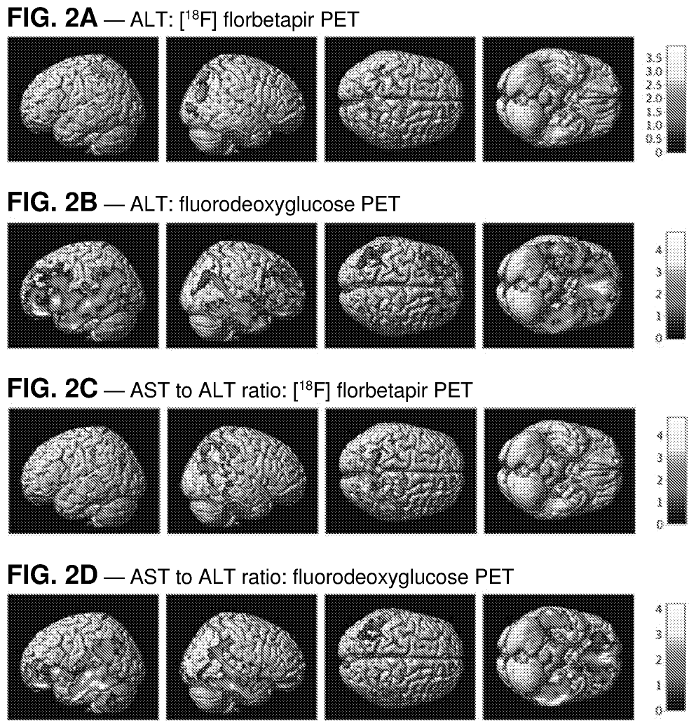

In the whole-brain analysis using multivariable regression models to determine the association of liver function markers with amyloid-β load measured from amyloid PET scans on a voxelwise level, significant associations for 2 liver function markers were identified. Higher ALT levels were significantly associated with reduced amyloid-β deposition in the bilateral parietal lobes (FIG. 2A). Increased AST to ALT ratio values were significantly associated with increased amyloid-β deposition in the bilateral parietal lobes and right temporal lobe (FIG. 2C).

### Biomarkers of Fibrillary Tau

CSF p-tau levels was used as a biomarker of fibrillary tau. The association of liver function markers with CSF p-tau was analyzed, adjusting for APOE ε4 status as a covariate. Higher AST to ALT ratio values were associated with higher CSF p-tau values (β[SE], 0.175 [0.055]; P=0.02) (FIG. 1).

### Biomarkers of Neurodegeneration or Neuronal Injury

Structural atrophy measured from MRI scans, brain glucose metabolism from FDG-PET scans, and CSF t-tau levels were used as biomarkers of neurodegeneration or neuronal injury.

### Brain Glucose Metabolism

An ROI-based association analysis was performed on liver function markers with a global cortical glucose metabolism value measured from FDG-PET scans across 1167 ADNI participants with both FDG-PET scans and measurement of liver function markers. The association analysis including APOE ε4 status as a covariate identified 2 markers as significantly associated with brain glucose metabolism after controlling for multiple testing using FDR (FIG. 1). For ALT, higher levels were associated with increased glucose metabolism (β[SE], 0.096 [0.030]; P=0.02), while for the AST to ALT ratio, higher ratio values were associated with reduced glucose metabolism (β[SE], −0.123 [0.042]; P=0.03).

In the detailed whole-brain analysis to determine the association of liver function markers with brain glucose metabolism on a voxelwise level, increased ALT levels were associated with increased glucose metabolism in a widespread pattern, especially in the bilateral frontal, parietal, and temporal lobes (FIG. 2B). However, higher AST to ALT ratio values were significantly associated with reduced glucose metabolism in the bilateral frontal, parietal, and temporal lobes (FIG. 2D).

### Structural MRI (Atrophy)

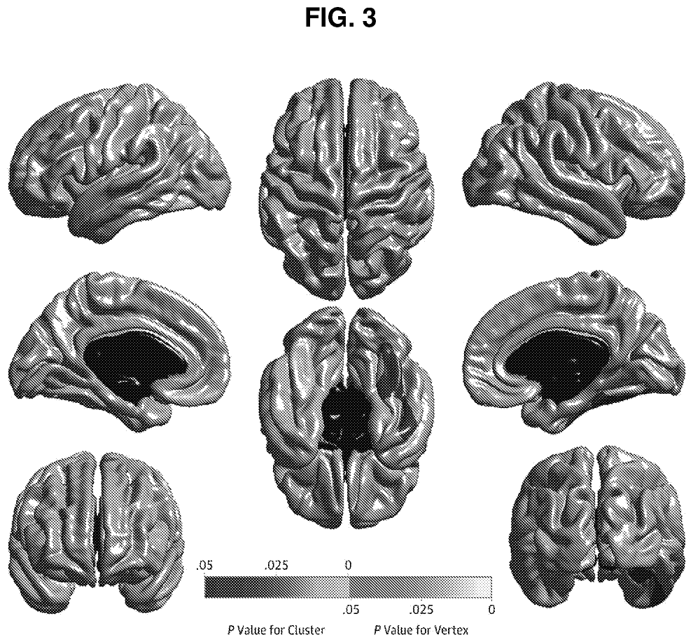

In the investigation of the association of liver function markers with mean hippocampal volume with APOE ε4 status as a covariate, no significant association with hippocampal volume was identified after controlling for multiple testing using FDR (FIG. 1). Following the detailed whole-brain surface-based analysis of liver function markers using multivariable regression models to assess associations with cortical thickness, higher ALT levels were significantly associated with larger cortical thickness in the bilateral temporal lobes (FIG. 3), which showed consistent patterns in the associations of brain glucose metabolism.

CSF t-Tau

Higher AST to ALT ratio values were associated with higher CSF t-tau levels (β[SE], 0.160 [0.049]; P=0.02) (FIG. 1), which showed consistent patterns in the associations of CSF amyloid-β1-42 or p-tau levels and brain glucose metabolism.

The association between serum-based liver function markers and AD diagnosis, cognition, and AD pathophysiological characteristics were investigated based on the A/T/N framework for AD biomarkers in the ADNI cohort [39]. These findings suggest that the decreased levels of ALT and elevated AST to ALT ratio that were observed in patients with AD were associated with poor cognition and reduced brain glucose metabolism. An increased AST to ALT ratio was associated with lower CSF amyloid-β 1-42 levels, greater amyloid-β deposition, and higher CSF p-tau and t-tau levels. Furthermore, decreased levels of ALT were associated with greater amyloid-β deposition and structural atrophy.

Decreased levels of ALT and increased AST to ALT ratio values were observed in patients with AD and were associated with lower scores on measures of memory and executive function. These findings are comparable with those of an earlier study that reported increased AST to ALT ratio values and lower levels of ALT in patients with AD compared with controls, although in that study, the association between AD and ALT levels did not reach statistical significance [40]. Altered liver enzymes lead to disturbances in liver-associated metabolites including branched-chain amino acids, ether-phosphatidylcholines, and lipids [41], which are altered in AD1 [42-44], and may play a role in disease pathophysiologic characteristics [45]. Disturbed energy metabolism is one of the processes that may explain the observed lower levels of ALT and increased enzyme ratio in individuals with AD and impaired cognition [3, 5]. This finding is concordant with the observation that increased AST to ALT ratio values and lower levels of ALT showed a consistent significant association with reduced brain glucose metabolism, particularly in the orbitofrontal cortex and temporal lobes, areas of the brain implicated in memory and executive function. Brain glucose hypometabolism is an early feature of AD and cognitive impairment during the prodromal stage [46, 47]. Moreover, ALT and AST are key enzymes in gluconeogenesis in the liver and production of neurotransmitters required in maintaining synapse [48]. Alanine aminotransferase catalyzes a reversible transamination reaction between alanine and α-ketoglutarate to form pyruvate and glutamate, while AST catalyzes a reversible reaction between aspartate and α-ketoglutarate to form oxaloacetate and glutamate [49]. See Schemes 1 and 2, above. Although exact mechanisms remain unclear [2], possible mechanisms may explain altered levels of enzymes in AD. First, reduced ALT levels lead to reduced pyruvate, which is required for glucose production via gluconeogenesis in the liver and glucose is distributed in various body tissues as an energy source [50], thus disturbing energy homeostasis. Second, altered levels of ALT and AST may affect levels of glutamate, an excitatory neurotransmitter of the central nervous system involved in synaptic transmission, which also plays an important role in memory [51],

In the case of low glucose metabolism in the brain, less α-ketoglutarate is available via the tricarboxylic acid cycle that favors glutamate catabolism versus glutamate synthesis in reversible reaction (catalyzed by AST and ALT) [52]. Glutamate acts as a neurotransmitter in approximately two-thirds of the synapses in neocortical and hippocampal pyramidal neurons and thus is involved in memory and cognition via long-term potentiation [53]. In a sample of healthy adults, plasma ALT and AST levels were significantly positively correlated with plasma glutamate levels [5, 54], which indicates that lower levels of ALT will decrease glutamate levels in plasma. Based on evidence from earlier studies that peripheral blood levels of glutamate are positively correlated with levels of glutamate in the CSF55 and studies that reported lower levels of glutamate in patients with AD compared with controls in both blood [56] and brain tissues, [36, 57-59] it is inferred that lower levels of ALT or AST may affect glutamate levels in AD. In older adults, lower serum ALT levels are associated with mortality [60, 61] and are thought to be a biomarker for increased frailty, sarcopenia, and/or reduced levels of pyridoxine (vitamin B6) [62]. Pyridoxine phosphate is a coenzyme for the synthesis of amino acids, neurotransmitters (e.g., serotonin and norepinephrine), and sphingolipids. Alanine aminotransferase decreases with age [63] and may be a sign of hepatic aging. Glutamate levels also decrease with increasing age [64]. Together with the fact that age is the strongest risk factor for AD [65], decreasing levels of ALT with age may also indicate a possible biological link between aging and AD. Nevertheless, further research is needed to determine the exact cause of reducing ALT levels with age and the pathway through which it can influence neurologic disorders, including AD.

Increased AST to ALT ratios are observed in individuals with nonalcoholic fatty liver disease, which is the hepatic manifestation of metabolic syndrome [66]. In the Framingham Heart Study, nonalcoholic fatty liver disease was associated with smaller total cerebral brain volume even after adjustment for multiple cardiovascular risk factors [67]. Liver dysfunction is also associated with the development of disease including cardiovascular disease and insulin resistance through disruptions in glucose and lipid metabolism, key physiological functions of the liver [68, 69]. Thus, using the AST to ALT ratio as a marker for overall metabolic disturbance [5], this study provides evidence of an association between altered metabolic status and AD, cognition, and AD endophenotypes.

In addition to ALT levels and the AST to ALT ratio, elevated levels of alkaline phosphatase were significantly associated with poor cognition. This is in line with results from the Oxford Project to Investigate Memory and Aging, which reported increased alkaline phosphatase levels in individuals with AD and an inverse association with cognition [70]. Alkaline phosphatase is an enzyme primarily expressed in the liver and kidneys as well as in endothelial cells in the brain [71, 72]. The neuronal form of alkaline phosphatase plays a role in developmental plasticity and activity-dependent cortical functions via contributing in γ-aminobutyric acid metabolism [73-76]. Changes in plasma levels of alkaline phosphatase may occur as a result of central nervous system injury [77].

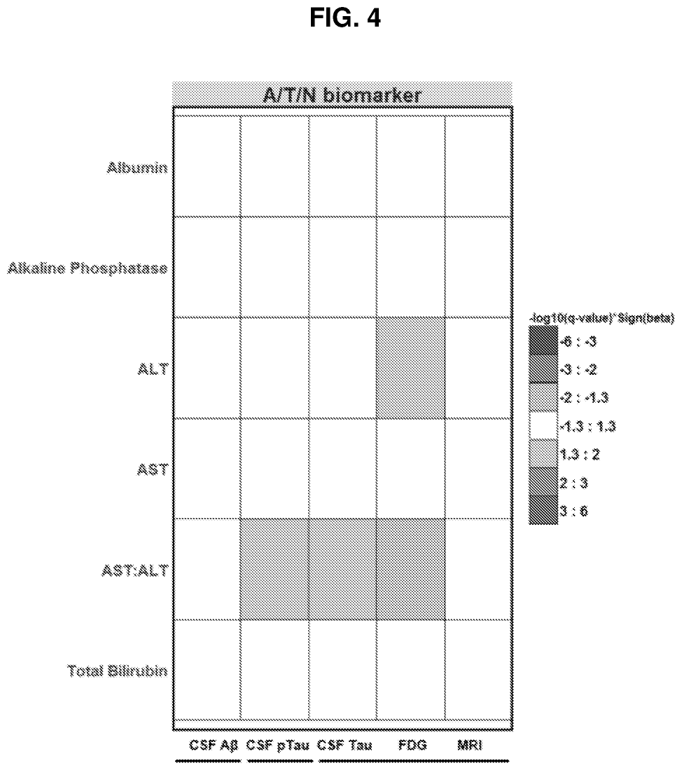

These results have several caveats. The observational design of this ADNI cohort study limits the ability to make assumptions about causality. There is need to evaluate the association of liver enzymes with AD in prospective manner. Another limitation of the study is that alcohol consumption was not adjusted for because it was not available in ADNI. Alcohol consumption is associated with altered liver enzymes. Instead, a well-established surrogate marker of alcohol consumption, γ-glutamyltransferase was used. Elevations in γ-glutamyltransferase generally indicate long-term heavy drinking rather than episodic heavy drinking [78]. The key findings remained significant after adjustment for γ-glutamyltransferase and statin use (Tables 6-7, and FIG. 4). However, given the associations with liver function measures and A/T/N biomarkers for AD, it appears that liver function may play a role in the pathogenesis of AD, but these limitations should be considered before further extrapolating the results.

TABLE 6
Diagnostic Group Differences of Liver Function
Biomarkers With Alzheimer Disease Diagnosis (CV vs. AD)
Adjusted for γ-Glutamyltransferase (GGT) and Statin Use

| | Odds | | Corrected |
|---|---|---|---|
| Liver Function Marker | Ratio | 95% Cl | P Value |
| Albumin (g/dL) | 5.595 | 0.037, 840.4 | 5.01 × 10⁻¹ |
| Alkaline Phosphatase (U/L) | 4.296 | 0.982, 18.79 | 1.06 × 10⁻¹ |
| ALT (U/L) | 0.112 | 0.032, 0.386 | 3.16 × 10⁻³ |
| AST (U/L) | 0.246 | 0.045, 1.348 | 1.59 × 10⁻¹ |
| AST:ALT | 8.580 | 1.713, 42.98 | 2.68 × 10⁻² |
| Total Bilirubin (mg/dL) | 1.461 | 0.603, 3.539 | 4.82 × 10⁻¹ |

Abbreviations: AST: Aspartate aminotransferase; ALT: alanine aminotransferase.

TABLE 7
Association of Liver Function Biomarkers With Cognition Adjusted
for γ-Glutamyltransferase (GGT) and Statin Use

| Liver | Memory | | | Executive function | | |
|---|---|---|---|---|---|---|
| Function | | | Corrected | | | Corrected |
| Marker | β | SE | P Value | β | SE | P Value |
| Albumin (g/dL) | −0.843 | 0.578 | 1.93 × 10⁻¹ | −0.238 | 0.691 | 7.31 × 10⁻¹ |
| Alkaline | −0.427 | 0.163 | 1.51 × 10⁻² | −0.607 | 0.194 | 5.52 × 10⁻³ |
| Phosphatase | | | | | | |
| (U/L) | | | | | | |
| ALT (U/L) | 0.476 | 0.139 | 3.62 × 10⁻³ | 0.733 | 0.165 | 1.18 × 10⁻⁴ |
| AST (U/L) | 0.366 | 0.187 | 7.62 × 10⁻² | 0.631 | 0.224 | 1.16 × 10⁻² |
| AST:ALT | −0.511 | 0.188 | 1.33 × 10⁻² | −0.709 | 0.224 | 5.52 × 10⁻³ |
| Total Bilirubin | −0.066 | 0.103 | 6.22 × 10⁻¹ | −0.070 | 0.123 | 6.22 × 10⁻¹ |
| (mg/dL) | | | | | | |

Covariates included age, sex, education, body mass index, APOE ε4 status, statins use, and γ-glutamyl transferase.
Abbreviations: AST: Aspartate aminotransferase; ALT: alanine aminotransferase; GGT: γ-glutamyl transferase.

This study's results suggest that altered liver function markers are associated with AD diagnosis and impaired memory and executive function as well as amyloid-β, tau, and neurodegenerative biomarkers of AD pathophysiological characteristics. These results are among the first to show an association of peripheral markers of liver functioning with central biomarkers associated with AD. Although these results suggest an important role of liver functioning in AD pathophysiological characteristics, the causal pathways remain unknown. The liver-brain biochemical axis of communication should be further evaluated in model systems and longitudinal studies to gain deeper knowledge of causal pathways.

### SUMMARY

Five serum-based liver function markers (total bilirubin, albumin, alkaline phosphatase, alanine aminotransferase, and aspartate aminotransferase) from AD Neuroimaging Initiative participants were used as exposure variables.

Primary outcomes included diagnosis of AD, composite scores for executive functioning and memory, CSF biomarkers, atrophy measured by magnetic resonance imaging, brain glucose metabolism measured by fludeoxyglucose, fluorine 18 (¹⁸F) positron emission tomography, and amyloid-β accumulation measured by [¹⁸F] florbetapir positron emission tomography.

Participants in the AD Neuroimaging Initiative (n=1581; 697 women and 884 men; mean [SD] age, 73.4 [7.2] years) included 407 cognitively normal older adults; 20 with significant memory concern; 298 with early mild cognitive impairment; 544 with late mild cognitive impairment; and 312 with AD. An elevated aspartate aminotransferase (AST) to alanine aminotransferase (ALT) ratio and lower levels of ALT were associated with AD diagnosis (AST to ALT ratio: odds ratio, 7.932 [95% Cl, 1.673-37.617]; P=0.03; ALT: odds ratio, 0.133 [95% Cl, 0.042-0.422]; P=0.004) and poor cognitive performance (AST to ALT ratio: β[SE], −0.465 [0.180]; P=0.02 for memory composite score; β[SE], −0.679 [0.215]; P=0.006 for executive function composite score; ALT: β[SE], 0.397 [0.128]; P=0.006 for memory composite score; β[SE], 0.637 [0.152]; P<0.001 for executive function composite score). Increased AST to ALT ratio values were associated with lower CSF amyloid-β 1-42 levels (β[SE], −0.170 [0.061]; P=0.04) and increased amyloid-β deposition (amyloid biomarkers), higher CSF phosphorylated tau₁₈₁, (β[SE], 0.175 [0.055]; P=0.02) (tau biomarkers) and higher CSF total tau levels (β[SE], 0.160 [0.049]; P=0.02) and reduced brain glucose metabolism (β[SE], −0.123 [0.042]; P=0.03) (neurodegeneration biomarkers). Lower levels of ALT were associated with increased amyloid-β deposition (amyloid biomarkers), and reduced brain glucose metabolism (β[SE], 0.096 [0.030]; P=0.02) and greater atrophy (neurodegeneration biomarkers).

Consistent associations of serum-based liver function markers with cognitive performance and A/T/N (amyloid, tau, and neurodegeneration) biomarkers for AD highlight the involvement of metabolic disturbances in the pathophysiology of AD. Liver enzyme involvement in AD opens avenues for novel diagnostics and therapeutics [79].

What is claimed:

1. A method for identifying or diagnosing Alzheimer's Disease (AD) or poor cognitive performance in a subject or population of subjects, the method comprising:
(a) detecting a concentration level in one or more samples from one or more subjects or a population of subjects of one or more biomarkers selected from alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, or a combination thereof,
wherein the level of ALT is less than the level in a control sample, or wherein the ratio of AST to ALT levels is greater than the ratio in a control sample;
(b) performing a genetic analysis on one or more samples from one or more subjects or a population of subjects to identify mutant, variant, or isozyme ALT, AST, or related genes; and
(c) diagnosing one or more subjects as having AD or poor cognitive performance based on the ALT level, AST level, or AST:ALT ratio determined in step (a), or the genetic analysis of the mutant, variant, or isozyme ALT, AST, or related genes identified in step (b).

2. A method for stratifying and determining the risk of one or more subjects of a population of subjects for developing Alzheimer's Disease (AD) or poor cognitive performance, the method comprising:
(a) detecting a concentration level in one or more samples from one or more subjects of a population of subjects of one or more biomarkers selected from alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, or bile acids,
wherein the level of ALT is less than the level in a control sample, or wherein the ratio of AST to ALT levels is greater than the ratio in a control sample;
(b) performing a genetic analysis on one or more samples from one or more subjects of a population of subjects to identify mutant, variant, or isozyme ALT, AST, or related genes;
(c) stratifying the levels of alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids,
phospholipids, bile acids, and genetic analyses of the mutant, variant, or isozyme ALT, AST, or related genes among the population of subjects to determining subjects at risk of developing AD or poor cognitive performance; and
(d) diagnosing subjects of the population of subject as at risk of developing or having AD or poor cognitive performance based on the stratification determined in step (c).

3. The method of claim 1 or 2, further comprising administering a treatment to the subject(s) determined to have a risk of developing or having AD or poor cognitive performance.

4. A method of treating Alzheimer's Disease (AD) or poor cognitive performance in a subject, or population of subjects, the method comprising:
(a) detecting a concentration level in one or more samples from one or more subjects or a population of subjects of one or more biomarkers selected from aspartate alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, or bile acids,
wherein the level of ALT is less than the level in a control sample, or wherein the ratio of AST to ALT levels is greater than the ratio in a control sample;
(b) performing a genetic analysis on one or more samples from one or more subjects or a population of subjects to identify mutant, variant, or isozyme ALT, AST, or related genes;
(c) diagnosing one or more subjects or population of subjects as having AD or poor cognitive performance based on the ALT level, AST level, or AST:ALT ratio determined in step (a), or the genetic analysis of the mutant, variant, or isozyme ALT, AST, or related genes identified in step (b); and
(d) administering a treatment to the subject(s) determined to have AD or poor cognitive performance.

5. The method of claim 4, wherein when a population of subjects are evaluated, the method further comprises step (b1) of stratifying the levels of alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, and the genetic analysis of the mutant, variant, or isozyme ALT, AST, or related genes among the population to determining subjects at risk of developing AD or poor cognitive performance.

6. The method of any one of claims 1-5, wherein the subject or population of subjects are further evaluated using clinical assays, magnetic resonance imaging (MRI), or position emission tomography (PET) for one or more of cerebral spinal fluid (CSF) amyloid-β1-42 levels; amyloid-β deposition; CSF phosphorylated tau levels; CSF total tau levels; brain glucose metabolism; brain atrophy, or a combination thereof.

7. The method of claim 6, wherein the subject or population of subjects diagnosed as having or at risk for AD or poor cognitive performance has one or more of: lower cerebral spinal fluid (CSF) amyloid-β1-42 levels; increased amyloid-β deposition; greater CSF phosphorylated tau levels; greater CSF total tau levels; reduced brain glucose metabolism; greater brain atrophy; or a combination thereof.

8. The method of claim 3 or 4, wherein the treatment comprises one or more of: bile acids (chenodeoxycholic acid (CDCA), cholic acid, ursodiol, tauroursodeoxycholic acid, ursodeoxycholic acid, obeticholic acid, glycocholic acid); bile acid sequestrants (Cholestyramine, Colesevelam, Colestilan, Colestipol, Ezetimibe); Statins (Atorvastatin, Lovastatin, Rosuvastatin, Simvastatin); fibrates (Fenofibrate); Heal Bile Acid Transporter (IBAT) inhibitors (Volixibat, Odevixibat, Elobixibat, Maralixibat, Albireo Pharma unnamed compounds); farnesoid X receptor agonists (Tropifexor, Cilofexor, EYP001a, GW4064, cafestol, chenodeoxycholic acid, obeticholic acid (OCA), Fexaramine, INT-767, Px-104, EDP-305, Gilead unnamed compounds; Metacrine unnamed compounds); G-protein-coupled bile acid receptor TRG5 agonists (6-EMCS, INT-777, Ardelyx unnamed compounds, Zydus Research Centre unnamed compounds); peroxisome proliferator-activated receptor (PPAR) agonists (Elafibranor GFT505); or Multidrug Resistance (MDR) Inhibitors (Vinblastine, Ritonavir, Furosemide, Lamivudine).

9. The method of any one of claim 3, 4, or 8, wherein the treatment comprises or further comprises one or more of: rivastigmine (Exelon®), galantamine (Razadyne®), memantine (Namenda®), a combination of memantine and donepezil (Namzaric®); antidepressants comprising citalopram (Celexa®), escitalopram (Lexapro®), fluoxetine (Prozac®), paroxetine (Paxil®), sertraline (Zoloft®), or trazodone (Desyrel®); anxiolytics comprising lorazepam (Ativan®) or oxazepam (Serax®); antipsychotic comprising aripiprazole (Abilify®), clozapine (Clozaril®), haloperidol (Haldol®), olanzapine (Zyprexa®), quetiapine (Seroquel®), risperidone (Risperdal®), or ziprasidone (Geodon®); tricyclic antidepressants comprising amitriptyline, amoxapine, desipramine (Norpramin®), doxepin, imipramine (Tofranil®), nortriptyline (Pamelor®), protriptyline, trimipramine; benzodiazepines comprising lorazepam, oxazepam or temazepam; sleeping treatments comprising zolpidem (Ambien®), zaleplon (Sonata®), eszopiclone (Lunesta®), phenobarbital, or chloral hydrate; atypical antipsychotics comprising risperidone, olanzapine, or quetiapine; classical antipsychotics comprising haloperidol; non-steroidal antiinflammatory drugs (NSAIDs, ibuprofen, naproxen, diclofenac, acetylsalicylic acid), acetaminophen, or alternative treatments or dietary supplements comprising amino acids (alanine, aspartate, glutamate, etc.), α-ketoglutarate, pyridoxal phosphate, vitamins (retinol (A), thiamine (B1), riboflavin (B2), niacinamide (B3), adenine (B4), pantothenic acid (B5), pyridoxine (B6), biotin (B7), adenylate (B8), carnitine (BT), folic acid (B9), cobalamin (B12), ascorbic acid (C), cholecalciferol (D), tocopherol (E), essential fatty acids (F), catechol (J), phylloquinone (K), salicylic acid (S), S-methylmethionine (U), inositol, choline), huperzine A, tramiprosate, caprylic acid, coconut oil, omega-3 fatty acids (fish oil, Lovaza®, Vascepa®, Epanova®, Omtryg®, Vscazen®), coenzyme Q10, phosphatidylserine, coral calcium, or Ginkgo biloba extracts.

10. The method of any one of claims 1-9, wherein the subject or population of subjects has liver disease.

11. The method of any one of claims 1-9, wherein the subject or population of subjects has decreased liver function.

12. The method of any one of claims 1-9, wherein the control sample is from a subject or population of subjects with normal cognition.

13. The method of any one of claims 1-9, wherein the control sample is from a subject or population of subjects not having AD or poor cognition.

14. The method of any one of claims 1-9, wherein the control sample is from a subject or population of subjects not having liver disease.

15. The method of any one of claims 1-14, wherein the sample comprises whole blood, serum, plasma, or cerebral spinal fluid (CSF).

16. The method of any one of claims 1-14, wherein the sample comprises blood.

17. The method of any one of claims 1-14, wherein the sample comprises cerebral spinal fluid (CSF).

18. Use of one or more biomarkers selected from alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, or a combination thereof and genetic analysis to identify mutant, variant, or isozyme ALT, AST, or related genes for identifying or diagnosing Alzheimer's Disease (AD) or poor cognitive performance in a subject or population of subjects.

19. Use of one or more biomarkers selected from alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, or a combination thereof and genetic analysis to identify mutant, variant, or isozyme ALT, AST, or related genes for stratifying a population of subjects for determining risk of developing or having Alzheimer's Disease (AD) or poor cognitive performance.

20. Use of one or more biomarkers selected from alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, or a combination thereof and genetic analysis to identify mutant, variant, or isozyme ALT, AST, or related genes for determining the risk of a subject or population of subjects developing Alzheimer's Disease (AD) or poor cognitive performance.

21. The use of any one of claims 18-20, further comprising administering a treatment to the subject determined to have AD or poor cognitive performance.

22. Use of one or more biomarkers selected from alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, or a combination thereof and genetic analysis to identify mutant, variant, or isozyme ALT, AST, or related genes for identifying or diagnosing Alzheimer's Disease (AD) or poor cognitive performance in a subject or population of subjects; and administering a treatment to the subject determined to have AD or poor cognitive performance.

23. The use of claim 22, wherein when a population of subjects are evaluated, the method further comprises step (b1) of stratifying the levels of alanine aminotransferase (ALT), aspartate aminotransferase (AST), ratio of AST to ALT levels, substrates and products related to enzymatic reactions catalyzed by ALT and AST, alkaline phosphatase, albumin, bilirubin, cholesterol and cholesterol metabolites, amino acids, phospholipids, bile acids, and genetic analyses of the mutant, variant, or isozyme ALT, AST, or related genes among the population to determining subjects at risk of developing AD or poor cognitive performance.

24. The use of any one of claims 18-23, wherein the subject or population of subjects are further evaluated using clinical assays, magnetic resonance imaging (MRI), or position emission tomography (PET) for one or more of cerebral spinal fluid (CSF) amyloid-β1-42 levels; amyloid-β deposition; CSF phosphorylated tau levels; CSF total tau levels; brain glucose metabolism; brain atrophy, or a combination thereof.

25. The use of claim 24, wherein the subject or population of subjects diagnosed as having or at risk for AD or poor cognitive performance has one or more of: lower cerebral spinal fluid (CSF) amyloid-β1-42 levels; increased amyloid-β deposition; greater CSF phosphorylated tau levels; greater CSF total tau levels; reduced brain glucose metabolism; greater brain atrophy; or a combination thereof.

26. The use of claim 21 or 22, wherein the treatment comprises one or more of: bile acids (chenodeoxycholic acid (CDCA), cholic acid, ursodiol, tauroursodeoxycholic acid, ursodeoxycholic acid, obeticholic acid, glycocholic acid); bile acid sequestrants (Cholestyramine, Colesevelam, Colestilan, Colestipol, Ezetimibe); Statins (Atorvastatin, Lovastatin, Rosuvastatin, Simvastatin); fibrates (Fenofibrate); Heal Bile Acid Transporter (IBAT) inhibitors (Volixibat, Odevixibat, Elobixibat, Maralixibat, Albireo Pharma unnamed compounds); farnesoid X receptor agonists (Tropifexor, Cilofexor, EYP001a, GW4064, cafestol, chenodeoxycholic acid, obeticholic acid (OCA), Fexaramine, INT-767, Px-104, EDP-305, Gilead unnamed compounds; Metacrine unnamed compounds); G-protein-coupled bile acid receptor TRG5 agonists (6-EMCS, INT-777, Ardelyx unnamed compounds, Zydus Research Centre unnamed compounds); peroxisome proliferator-activated receptor (PPAR) agonists (Elafibranor GFT505); or Multidrug Resistance (MDR) Inhibitors (Vinblastine, Ritonavir, Furosemide, Lamivudine).

27. The use of any one of claim 21, 22, or 26, wherein the treatment comprises or further comprises one or more of: rivastigmine (Exelon®), galantamine (Razadyne®), memantine (Namenda®), a combination of memantine and donepezil (Namzaric®); antidepressants comprising citalopram (Celexa®), escitalopram (Lexapro®), fluoxetine (Prozac®), paroxetine (Paxil®), sertraline (Zoloft®), or trazodone (Desyrel®); anxiolytics comprising lorazepam (Ativan®) or oxazepam (Serax®); antipsychotic comprising aripiprazole (Abilify®), clozapine (Clozaril®), haloperidol (Haldol®), olanzapine (Zyprexa®), quetiapine (Seroquel®), risperidone (Risperdal®), or ziprasidone (Geodon®); tricyclic antidepressants comprising amitriptyline, amoxapine, desipramine (Norpramin®), doxepin, imipramine (Tofranil®), nortriptyline (Pamelor®), protriptyline, trimipramine; benzodiazepines comprising lorazepam, oxazepam or temazepam; sleeping treatments comprising zolpidem (Ambien®), zaleplon (Sonata®), eszopiclone (Lunesta®), phenobarbital, or chloral hydrate; atypical antipsychotics comprising risperidone, olanzapine, or quetiapine; classical antipsychotics comprising haloperidol; non-steroidal antiinflammatory drugs (NSAIDs, ibuprofen, naproxen, diclofenac, acetylsalicylic acid), acetaminophen, or alternative treatments or dietary supplements comprising amino acids (alanine, aspartate, glutamate, etc.), α-ketoglutarate, pyridoxal phosphate, vitamins (retinol (A), thiamine (B1), riboflavin (B2), niacinamide (B3), adenine (B4), pantothenic acid (B5), pyridoxine (B6), biotin (B7), adenylate (B8), carnitine (BT), folic acid (B9), cobalamin (B12), ascorbic acid (C), cholecalciferol (D), tocopherol (E), essential fatty acids (F), catechol (J), phylloquinone (K), salicylic acid (S), S-methylmethionine (U), inositol, choline), huperzine A, tramiprosate, caprylic acid, coconut oil, omega-3 fatty acids (fish oil, Lovaza®, Vascepa®, Epanova®, Omtryg®, Vscazen®), coenzyme Q10, phosphatidylserine, coral calcium, or Ginkgo biloba extracts.

28. The use of any one of claims 18-27, wherein the subject or population of subjects has liver disease.

29. The use of any one of claims 18-27, wherein the subject or population of subjects has decreased liver function.

30. The use of any one of claims 18-27, wherein the control sample is from a subject or population of subjects with normal cognition.

31. The use of any one of claims 18-27, wherein the control sample is from a subject or population of subjects not having AD or poor cognition.

32. The use of any one of claims 18-27, wherein the control sample is from a subject or population of subjects not having liver disease.

33. The use of any one of claims 18-27, wherein the sample comprises whole blood, serum, plasma, or cerebral spinal fluid (CSF).

34. The use of any one of claims 18-27, wherein the sample comprises blood.

35. The use of any one of claims 18-27, wherein the sample comprises cerebral spinal fluid (CSF).


SEQUENCE LISTING (4 sequences)

SEQ ID NO: 1 - DNA, 1491 bp, Homo sapiens
  CDS (1)..(1491)
atggcctcgagcacaggtgaccggagccaggcggtgaggcatggactgagggcgaaggtgctgacgctggacggcatgaacccgcgtgtgcggagagtggagtacgcagtgcgtggccccatagtgcagcgagccttggagctggagcaggagctgcgccagggtgtgaagaagcctttcaccgaggtcatccgtgccaacatcggggacgcacaggctatggggcagaggcccatcaccttcctgcgccaggtcttggccctctgtgttaaccctgatcttctgagcagccccaacttccctgacgatgccaagaaaagggcggagcgcatcttgcaggcgtgtgggggccacagtctgggggcctacagcgtcagctccggcatccagctgatccgggaggacgtggcgcggtacattgagaggcgtgacggaggcatccctgcggaccccaacaacgtcttcctgtccacaggggccagcgatgccatcgtgacggtgctgaagctgctggtggccggcgagggccacacacgcacgggtgtgctcatccccatcccccagtacccactctactcggccacgctggcagagctgggcgcagtgcaggtggattactacctggacgaggagcgtgcctgggcgctggacgtggccgagcttcaccgtgcactgggccaggcgcgtgaccactgccgccctcgtgcgctctgtgtcatcaaccctggcaaccccaccgggcaggtgcagacccgcgagtgcatcgaggccgtgatccgcttcgccttcgaagagcggctctttctgctggcggacgaggtgtaccaggacaacgtgtacgccgcgggttcgcagttccactcattcaagaaggtgctcatggagatggggccgccctacgccgggcagcaggagcttgcctccttccactccacctccaagggctacatgggcgagtgcgggttccgcggcggctatgtggaggtggtgaacatggacgctgcagtgcagcagcagatgctgaagctgatgagtgtgcggctgtgcccgccggtgccaggacaggccctgctggacctggtggtcagcccgcccgcgcccaccgacccctcctttgcgcagttccaggctgagaagcaggcagtgctggcagagctggcggccaaggccaagctcaccgagcaggtcttcaatgaggctcctggcatcagctgcaacccagtgcagggcgccatgtactccttcccgcgcgtgcagctgcccccgcgggcggtggagcgcgctcaggagctgggcctggcccccgatatgttcttctgcctgcgcctcctggaggagaccggcatctgcgtggtgccagggagcggctttgggcagcgggaaggcacctaccacttccggatgaccattctgccccccttggagaaactgcggctgctgctggagaagctgagcaggttccatgccaagttcaccctcgagtactcctga

SEQ ID NO: 2 - protein, 496 aa, Homo sapiens
MASSTGDRSQAVRHGLRAKVLTLDGMNPRVRRVEYAVRGPIVQRALELEQELRQGVKKPFTEVIRANIGDAQAMGQRPITFLRQVLALCVNPDLLSSPNFPDDAKKRAERILQACGGHSLGAYSVSSGIQLIREDVARYIERRDGGIPADPNNVFLSTGASDAIVTVLKLLVAGEGHTRTGVLIPIPQYPLYSATLAELGAVQVDYYLDEERAWALDVAELHRALGQARDHCRPRALCVINPGNPTGQVQTRECIEAVIRFAFEERLFLLADEVYQDNVYAAGSQFHSFKKVLMEMGPPYAGQQELASFHSTSKGYMGECGFRGGYVEVVNMDAAVQQQMLKLMSVRLCPPVPGQALLDLVVSPPAPTDPSFAQFQAEKQAVLAELAAKAKLTEQVFNEAPGISCNPVQGAMYSFPRVQLPPRAVERAQELGLAPDMFFCLRLLEETGICVVPGSGFGQREGTYHFRMTILPPLEKLRLLLEKLSRFHAKFTLEYS

SEQ ID NO: 3 - DNA, 882 bp, Homo sapiens
  CDS (1)..(882)
atggcacctccgtcagtctttgccgaggttccgcaggcccagcctgtcctggtcttcaagctcactgccgacttcagggaggatccggacccccgcaaggtcaacctgggagtgggagcatatcgcacggatgactgccatccctgggttttgccagtagtgaagaaagtggagcagaagattgctaatgacaatagcctaaatcacgagtatctgccaatcctgggcctggctgagttccggagctgtgcttctcgtcttgcccttggggatgacagcccagcactcaaggagaagcgggtaggaggtgtgcaatctttggggggaacaggtgcacttcgaattggagctgatttcttagcgcgttggtacaatggaacaaacaacaagaacacacctgtctatgtgtcctcaccaacctgggagaatcacaatgctgtgttttccgctgctggttttaaagacattcggtcctatcgctactgggatgcagagaagagaggattggacctccagggcttcctgaatgatctggagaatgctcctgagttctccattgttgtcctccacgcctgtgcacacaacccaactgggattgacccaactccggagcagtggaagcagattgcttctgtcatgaagcaccggtttctgttccccttctttgactcagcctatcagggcttcgcatctggaaacctggagagagatgcctgggccattcgctattttgtgtctgaaggcttcgagttcttctgtgcccagtccttctccaagaacttcgggctctacagtgagtgctctcctgaggagttacagccccacctcctcgcatcactccctgcttcagcagccacagctgattctggtccctctctttag

SEQ ID NO: 4 - protein, 293 aa, Homo sapiens
MAPPSVFAEVPQAQPVLVFKLTADFREDPDPRKVNLGVGAYRTDDCHPWVLPVVKKVEQKIANDNSLNHEYLPILGLAEFRSCASRLALGDDSPALKEKRVGGVQSLGGTGALRIGADFLARWYNGTNNKNTPVYVSSPTWENHNAVFSAAGFKDIRSYRYWDAEKRGLDLQGFLNDLENAPEFSIVVLHACAHNPTGIDPTPEQWKQIASVMKHRFLFPFFDSAYQGFASGNLERDAWAIRYFVSEGFEFFCAQSFSKNFGLYSECSPEELQPHLLASLPASAATADSGPSL